(19) United States
(12) Patent Application Publication
(10) Pub. No.: US 2023/0309564 A1
(43) Pub. Date: Oct. 5, 2023

(54) METHYLOBACTERIUM STRAINS FOR IMPROVING PRODUCTION AND QUALITY OF PLANTS AND METHODS RELATED THERETO

(71) Applicant: NewLeaf Symbiotics, Inc., St. Louis, MO (US)

(72) Inventors: Patrick Vogan, St. Louis, MO (US); Janne Kerovuo, St. Louis, MO (US); Natalie Breakfield, St. Louis, MO (US)

(21) Appl. No.: 18/000,590

(22) PCT Filed: Jun. 2, 2021
(86) PCT No.: PCT/US2021/035480

§ 371 Date: Dec. 2, 2022

Related U.S. Application Data
(60) Provisional application No. 63/088,837, filed on Oct. 7, 2020.
(60) Provisional application No. 63/033,364, filed on Jun. 2, 2020.

(51) Int. Cl. A01N 63/20; A01P 21/00; A01H 3/00; C12N 1/20

(52) U.S. Cl. CPC A01N 63/20; A01H 3/00; A01P 21/00; C12N 1/20; C12R 2001/01

(57) ABSTRACT

Methylobacterium strains that enhance early plant growth and methods of their use are provided herein. Also provided are methods for identifying Methylobacterium strains which can be used to increase the content of one or more mineral nutrient and/or vitamins in a leafy green plant are provided. Also provided are related methods of providing leafy green plants with increased levels of one or more mineral nutrients and/or vitamins, and leafy green plants and harvested greens from said plants having increased levels of one or more mineral nutrients and/or vitamins, as the result of treatment with Methylobacterium strains as provided herein.

### CROSS-REFERENCE TO RELATED APPLICATIONS

This international patent application claims the benefit of U.S. Provisional Pat. Application No. 63/033,364, filed Jun. 2, 2020 and U.S. Provisional Pat. Application No. 63/088,837, filed Oct. 7, 2020, the entire disclosures of which are incorporated herein by reference.

### SEQUENCE LISTING STATEMENT

A sequence listing containing the file named “LeafyGreens_ST25.txt” which is 22,944 bytes (measured in MS-Windows®) and created on May 25, 2021, contains 76 nucleic acid sequences is provided herewith via the USPTO’s EFS system, and is incorporated herein by reference in its entirety.

### BACKGROUND

Plants require certain macronutrients and micronutrients for growth and metabolism. These elements are generally found in the soil as salts and can be consumed by plants as ions. In agriculture, soil can become depleted of one or more of these nutrients requiring the addition of fertilizers to provide sufficient quantities of the nutrients for crop growth. In hydroponic systems, all nutrients must be supplied to the growing plants and are often the greatest cost for a hydroponic plant production system. Plants are an important part of a healthy diet, and leafy greens in particular, are known for their high content of vitamins and nutrients. Methods of enhancing production by improving growth and/or increasing levels of macronutrients and micronutrients in plants are desired for benefits to agricultural practices and to human and animal nutrition.

One-carbon organic compounds such as methane and methanol are found extensively in nature, and are utilized as carbon sources by bacteria classified as methanotrophs and methylotrophs. Methanotrophic bacteria include species in the genera Methylobacter, Methylomonas, Methylomicrobium, Methylococcus, Methylosinus, Methylocystis, Methylosphaera, Methylocaldum, and Methylocella (Lidstrom, 2006). Methanotrophs possess the enzyme methane monooxygenase which incorporates an atom of oxygen from O₂ into methane, forming methanol. All methanotrophs are obligate one-carbon utilizers that are unable to use compounds containing carbon-carbon bonds. Methylotrophs, on the other hand, can also utilize more complex organic compounds, such as organic acids, higher alcohols, sugars, and the like. Thus, methylotrophic bacteria are facultative methylotrophs. Methylotrophic bacteria include species in the genera Methylobacterium, Hyphomicrobium, Methylophilus, Methylobacillus, Methylophaga, Aminobacter, Methylorhabdus, Methylopila, Methylosulfonomonas, Marinosulfonomonas, Paracoccus, Xanthobacter, Ancylobacter (also known as Microcyclus), Thiobacillus, Rhodopseudomonas, Rhodobacter, Acetobacter, Bacillus, Mycobacterium, Arthobacter, and Nocardia (Lidstrom, 2006).

Some methylotrophic bacteria of the genus Methylobacterium are pink-pigmented. They are conventionally referred to as PPFM bacteria, being pink-pigmented facultative methylotrophs. Green (2005, 2006) identified twelve validated species in the genus Methylobacterium, specifically M. aminovorans, M. chloromethanicum, M. dichloromethanicum, M. extorquens, M. fujisawaense, M. mesophilicum, M. organophilum, M. radiotolerans, M. rhodesianum, M. rhodinum, M. thiocyanatum, and M. zatmanii. However, M. nodulans is a nitrogen-fixing Methylobacterium that is not a PPFM (Sy et al., 2001). Methylobacterium are found in soil, dust, fresh water, sediments, and leaf surfaces, as well as in industrial and clinical environments (Green, 2006).

### SUMMARY

Provided herein are compositions comprising one or more Methylobacterium strains that enhance early growth of plants, improve propagation/transplant vigor, increase nutrient uptake, improve stand establishment, and/or improve stress tolerance. In certain embodiments, the Methylobacterium in the composition is selected from the group consisting of LGP2022, LGP2023 and LGP2021. In certain embodiments, the Methylobacterium in the composition is a variant of LGP2022, LGP2023 or LGP2021. In certain embodiments, the plants are leafy green plants, including microgreens and/or herbs. In certain embodiments, the plants are fruit or vegetable plants. In certain embodiments, the plants are agricultural row crops. In certain embodiments, the plants are grown in a greenhouse. In certain embodiments, the plants are grown hydroponically or aeroponically. Also provided is an isolated Methylobacterium selected from LGP2022, LGP2023 and LGP2021, compositions comprising such Methylobacterium isolates or variants thereof, and plants, plant parts or seeds that are at least partially coated with compositions comprising LGP2022, LGP2023, LGP2021, or variants thereof.

Methods of improving the production of plants by applying one or more Methylobacterium strains to the plant, a plant part, or to a seed are provided herein. In some embodiments, the composition comprising one or more Methylobacterium strains is applied such that it coats or partially coats the plant, plant part, or seed. In some embodiments, plant production is improved by enhancing early plant growth. In some embodiments, plant production is improved by increasing rooting of the plant. In some embodiments, plant production is improved by increasing the content of nutrients present in the plant or a plant part. In some embodiments, plant production is improved by enhancing propagation/transplant vigor. In some embodiments, plant production is improved by enhancing stand establishment. In some embodiments, plant production is improved by enhancing stress tolerance. In certain embodiments, the Methylobacterium in the composition is selected from the group consisting of LGP2009, LGP2002, LGP2019, LGP2022, LGP2023 and LGP2021. In some embodiments, the Methylobacterium in the composition is selected from the group consisting of LGP2001, LGP2002, LGP2009, LGP2015, a combination of LGP2002 and LGP2015, and variants thereof and the composition is applied such that it coats or partially coats the plant, plant part, or seed, wherein the plant comprises rosemary, French tarragon, basil, Pennisetum, and/or other herbs. In certain embodiments, the Methylobacterium in the composition is a variant of any of the aforementioned Methylobacterium isolates. In certain embodiments, the plants are leafy green plants. In certain embodiments, plant biomass is increased by treatment with one or more Methylobacterium strains as provided herein. In some embodiments, plant biomass is increased as the result of enhanced early growth resulting from treatment with LGP2022, LGP2023 or LGP2021, or variants thereof. In some embodiments, enhanced early growth is assessed at the two true leaf stage of development. In certain embodiments of the methods provided herein, the Methylobacterium compositions are applied to plants, plant parts or seeds of fruits or vegetables grown hydroponically. In some embodiments, the Methylobacterium compositions provided herein are applied to plants, plant parts or seeds of leafy green vegetables. In some embodiments, such leafy green vegetables are grown hydroponically. In certain embodiments, the plants are agricultural row crops.

In certain embodiments of methods to improve plant production provided herein, the plant is a leafy green plant, the plant improvement comprises enhanced early growth, increased levels of nutrients, improved propagation/transplant vigor, improved stand establishment, and/or improved stress tolerance in the plant or plant part and the Methylobacterium is selected from LGP2009, LGP2022, LGP2023 or LGP2021, or variants thereof. In some embodiments, the leafy green plant is selected from the group consisting of spinach, lettuce, beets, swiss chard, watercress, kale, collards, escarole, arugula, endive, bok choy, and turnips. In some embodiments, the Methylobacterium is selected from LGP2001, LGP2002, LGP2009, LGP2015, a combination of LGP2002 and LGP2015, and variants thereof and the leafy green plant comprises rosemary, French tarragon, basil, Pennisetum, and/or other herbs. In certain embodiments of methods to improve plant production provided herein, the plant is a cannabis plant, the plant improveme×nt is selected from enhanced growth and/or rooting, decreased cycling time and increased biomass or yield and the Methylobacterium is selected from LGP2002, LGP2009, LGP2019 and variants thereof. In certain embodiments, a variant of LGP2002 has genomic DNA comprising one or more polynucleotide marker fragments of at least 50, 60, 100, 120, 180, 200, 240, or 300 nucleotides of SEQ ID NOS: 13-15. In certain embodiments, a variant of LGP2009 has genomic DNA comprising one or more polynucleotide marker fragments of at least 50, 60, 100, 120, 180, 200, 240, or 300 nucleotides of SEQ ID NOS: 71-73. In certain embodiments, a variant of LGP2019 has genomic DNA comprising one or more polynucleotide marker fragments of at least 50, 60, 100, 120, 180, 200, 240, or 300 nucleotides of SEQ ID NOS: 25-27.

In certain embodiments of the compositions and methods provided herein, the composition further comprises at least one additional component selected from the group consisting of an additional active ingredient, an agriculturally acceptable adjuvant, and an agriculturally acceptable excipient. In certain embodiments of any of the aforementioned methods, the composition comprises the Methylobacterium at a titer of greater than 1×10³ CFU/gm or at a titer of about 1×10⁶ CFU/gm to about 1×10¹⁴ CFU/gm for a solid composition or at a titer of greater than 1×10³ CFU/ml or at a titer of about 1×10⁶ CFU/mL to about 1×10¹¹ CFU/mL for a liquid composition.

Also provided herein are methods of improving growth and yield of rice plants by treating rice plants, plant parts or seeds with one or more Methylobacterium isolates. In some embodiments, harvested seed yield and/or nutrient content of rice plants is improved. In some embodiments, rice seeds are treated and such treatment provides for increased rice seed yield. In some embodiments, the Methylobacterium isolate is selected from the group consisting of LGP2016 (ISO117), LGP2017 (ISO118), LGP2019 (ISO120) and variants of these isolates. Rice plants, plant parts or seeds coated with Methylobacterium isolates and/or compositions are also provided herein. In certain embodiments, the Methylobacterium has chromosomal genomic DNA having at least 99%, 99.9, 99.8, 99.7, 99.6%, or 99.5% sequence identity to chromosomal genomic DNA of LGP2016, LGP2017, or LGP2019. In certain embodiments, the Methylobacterium has genomic DNA comprising one or more polynucleotide marker fragments of at least 50, 60, 100, 120, 180, 200, 240, or 300 nucleotides of SEQ ID NOS: 37-39 or SEQ ID NOS: 25-27.

Also provided herein are methods of improving growth and production of cannabis plants by treating cannabis plants, plant parts or seeds with one or more Methylobacterium isolates. In some embodiments, nutrient content of treated plants is improved. In some embodiments, a cannabis cutting from a mature plant is treated. In some embodiments, such treatments improve plant growth and rooting of such cuttings. In some embodiments, such treatments provided for a decreased cycling time for production of a cannabis plant as the result of such increased plant growth and rooting. In some embodiments, the Methylobacterium isolate is selected from the group consisting of LGP2002, LGP2009, LGP2019 and variants of these isolates. Cannabis plants, plant parts or seeds coated with Methylobacterium isolates and/or compositions are also provided herein. In certain embodiments, the Methylobacterium has chromosomal genomic DNA having at least 99%, 99.9, 99.8, 99.7, 99.6%, or 99.5% sequence identity to chromosomal genomic DNA of LGP2002, LGP2009, or LGP2019. In certain embodiments, the Methylobacterium has genomic DNA comprising one or more polynucleotide marker fragments of at least 50, 60, 100, 120, 180, 200, 240, or 300 nucleotides of SEQ ID NOS: 13-15, SEQ ID NOS: 71-73 or SEQ ID NOS: 25-27.

Also provided herein are methods for identifying a Methylobacterium isolate that increases the content of at least one mineral nutrient and/or at least one vitamin in a leafy green plant or plant part comprising: (i) treating a leafy green plant seed and/or a leafy green plant with at least a first Methylobacterium strain to obtain a treated seed and/or a treated plant; (ii) harvesting a plant part from a cultivated plant wherein the cultivated plant is grown from the treated seed or treated plant of step (i); (ii) harvesting a plant part from a cultivated control plant wherein the cultivated control plant was grown from an untreated control seed or untreated control plant; (iii) determining the content of at least one mineral nutrient and/or vitamin in the plant part from the cultivated plant and from the cultivated control plant; and, (iv) selecting a Methylobacterium strain that increases the content of at least one mineral nutrient or vitamin in the cultivated plant or a plant part of the cultivated plant in comparison to the content of the at least one mineral nutrient or vitamin in the cultivated control plant or plant part.

Methods for identifying a Methylobacterium isolate that increases the content of one or more mineral nutrients and/or vitamins, by separately treating and analyzing plants with two or more Methylobacterium isolates are also provided herein. Such methods comprise: (i) treating a first leafy green plant seed or plant with at least a first Methylobacterium strain and a second leafy green plant seed or plant part with a second Methylobacterium strain, (ii) harvesting a plant part from a plant grown from the first seed or plant, from a plant grown from the second seed or plant, and optionally from a plant grown from an untreated control seed or from an untreated control plant; (iii) analyzing a plant part harvested from the plant grown from the first seed or plant, from the plant grown from the second seed or plant, and optionally from the plant grown from the control seed or plant to determine the content of at least one mineral nutrient and/or vitamin, and (iv) selecting the Methylobacterium strain that provides the greatest increase in the content of the at least one mineral nutrient and/or vitamin. Such methods can also be used to test three or more Methylobacterium isolates. In such methods, seeds and/or plants are separately treated with three or more distinct Methylobacterium isolates or combinations of distinct Methylobacterium isolates, and said treated seeds or seedlings are separately cultivated, harvested and analyzed to determine the mineral nutrient and/or vitamin content in the plants, shoots or one or more plant leaves in comparison to the mineral nutrient and/or vitamin content in other distinct Methylobacterium treatments and, optionally, to an untreated control plant.

Methods of producing a leafy green food product with increased levels of one or more mineral nutrients and/or vitamins comprising harvesting leafy greens from a cultivated plant or plants grown from Methylobacterium-treated seeds, plants or plant parts, thereby obtaining leafy greens with increased levels of one or more mineral nutrients and/or vitamins are also provided. In certain embodiments of such methods, the Methylobacterium strain is LGP2009 (ISO110 (NRRL B-50938)) or a variant thereof. In certain embodiments, the Methylobacterium strain has chromosomal genomic DNA having at least 99%, 99.9, 99.8, 99.7, 99.6%, or 99.5% sequence identity to chromosomal genomic DNA of ISO110 (NRRL B-50938). In certain embodiments, the Methylobacterium strain has genomic DNA comprising one or more polynucleotide marker fragments of at least 50, 60, 100, 120, 180, 200, 240, or 300 nucleotides of SEQ ID NOS: 71-73. In certain embodiments of the aforementioned methods, the composition comprises (i) a Methylobacterium wherein the assembled genome DNA sequence of the Methylobacterium has an average nucleotide identity (ANI) score of at least 99.00 when compared to the assembled genome DNA sequence of ISO110 (NRRL B-50938). Methods of producing a harvested leafy green food product with increased levels of one or more mineral nutrients and/or vitamins comprising harvesting leafy greens from leafy green plants grown from seeds and/or seedlings treated with an effective amount of a Methylobacterium strain, wherein said leafy green plants are mature plants, immature plants having from two to four true leaves, or immature plants having less than two true leaves, thereby obtaining a leafy green, baby green or micro green food product with increased levels of one or more mineral nutrients and/or vitamins are provided. In certain embodiments of such methods, the Methylobacterium is ISO110 (NRRL B-50938. In certain embodiments, the Methylobacterium has chromosomal genomic DNA having at least 99%, 99.9, 99.8, 99.7, 99.6%, or 99.5% sequence identity to chromosomal genomic DNA of ISO110 (NRRL B-50938). In certain embodiments, the Methylobacterium has genomic DNA comprising one or more polynucleotide marker fragments of at least 50, 60, 100, 120, 180, 200, 240, or 300 nucleotides of SEQ ID NOS: 71-73. In certain embodiments, the leafy green plants are cultivated in a hydroponic system. In certain embodiments, the leafy green plants are spinach plants.

A leafy green plant, or plant part having increased levels of one or more mineral nutrients and/or vitamins is also provided herein. Said leafy green plant or plant part is harvested from a cultivated plant grown from a Methylobacterium-treated seed, plant or plant part, wherein said Methylobacterium provides for increased levels of one or more mineral nutrients and/or vitamins. In certain embodiments, the Methylobacterium is ISO110 (NRRL B-50938). In certain embodiments, the Methylobacterium has chromosomal genomic DNA having at least 99%, 99.9, 99.8, 99.7, 99.6%, or 99.5% sequence identity to chromosomal genomic DNA of ISO110 (NRRL B-50938). In certain embodiments, the Methylobacterium has genomic DNA comprising one or more polynucleotide marker fragments of at least 50, 60, 100, 120, 180, 200, 240, or 300 nucleotides of SEQ ID NOS: 71-73. In certain embodiments, said Methylobacterium comprises a Methylobacterium wherein the assembled genome DNA sequence of the Methylobacterium has an average nucleotide identity (ANI) score of at least 99.00 when compared to the assembled genome DNA sequence of ISO110 (NRRL B-50938). In certain embodiments, the leafy green plants are spinach plants.

### DETAILED DESCRIPTION

### Definitions

The term “and/or” where used herein is to be taken as specific disclosure of each of the two or more specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

As used herein, the terms “include,” “includes,” and “including” are to be construed as at least having the features or encompassing the items to which they refer while not excluding any additional unspecified features or unspecified items.

As used herein, the term “biological” refers to a component of a composition for treatment of plants or plant parts comprised of or derived from a microorganism. Biologicals include biocontrol agents, other beneficial microorganisms, microbial extracts, natural products, plant growth activators or plant defense agents. Non-limiting examples of biocontrol agents include bacteria, fungi, beneficial nematodes, and viruses. In certain compositions, a biological can comprise a mono-culture or co-culture of Methylobacterium, or a combination of Methylobacterium strains or isolates that have been separately cultured.

As used herein, a “leafy green plant” refers to a vegetable crop with edible leaves and includes, without limitation, spinach, kale, lettuce (including but not limited to romaine, butterhead, iceberg and loose leaf lettuces), collard greens, cabbage, beet greens, watercress, swiss chard, arugula, escarole, endive, bok choy and turnip greens. Leafy green plants as used herein also refers to plants grown for harvest of microgreens and/or herbs, including but not limited to lettuce, cauliflower, broccoli, cabbage, watercress, arugula, garlic, onion, leek, amaranth, swill chard, been, spinach, melon, cucumber, squash, basil, celery, cilantro, radish, radicchio, chicory, dill, rosemary, French tarragon, basil, Pennisetum, carrot, fennel, beans, peas, chickpeas, and lentils. Leafy green plants also refer to mixes of assorted leafy green plants, such as mesclun or other mixed salad greens or mixed microgreens. “Leafy green plants” as used herein also encompasses other brassica or cruciferous field greens not specifically mentioned herein by name.

As used herein, a “fruit” or “fruit bearing plant” is a fleshy fruit bearing plant, including but not limited to, melon (including watermelon and cantaloupe), berry (including strawberry, blueberry, blackberry and raspberry), grape, kiwi, mango, papaya, pineapple, banana, pepper, tomato, squash, and cucumber plants.

As used herein, the term “Methylobacterium” refers to genera and species in the methylobacteriaceae family, including bacterial species in the Methylobacterium genus and proposed Methylorubrum genus (Green and Ardley (2018)). Methylobacterium includes pink-pigmented facultative methylotrophic bacteria (PPFM) and also encompasses the non-pink-pigmented Methylobacterium nodulans, as well as colorless mutants of Methylobacterium isolates. For example, and not by way of limitation, “Methylobacterium” refers to bacteria of the species listed below as well as any new Methylobacterium species that have not yet been reported or described that can be characterized as Methylobacterium or Methylorubrum based on phylogenetic analysis: Methylobacterium adhaesivum; Methylobacterium oryzae; Methylobacterium aerolatum; Methylobacterium oxalidis; Methylobacterium aquaticum; Methylobacterium persicinum; Methylobacterium brachiatum; Methylobacterium phyllosphaerae; Methylobacterium brachythecii; Methylobacterium phyllostachyos; Methylobacterium bullatum; Methylobacterium platani; Methylobacterium cerastii; Methylobacterium pseudosasicola; Methylobacterium currus; Methylobacterium radiotolerans; Methylobacterium dankookense; Methylobacterium soli; Methylobacterium frigidaeris; Methylobacterium specialis; Methylobacterium fujisawaense; Methylobacterium tardum; Methylobacterium gnaphalii; Methylobacterium tarhaniae; Methylobacterium goesingense; Methylobacterium thuringiense; Methylobacterium gossipiicola; Methylobacterium trifolii; Methylobacterium gregans; Methylobacterium variabile; Methylobacterium haplocladii; Methylobacterium aminovorans (Methylorubrum aminovorans); Methylobacterium hispanicum; Methylobacterium extorquens (Methylorubrum extorquens); Methylobacterium indicum; Methylobacterium podarium (Methylorubrum podarium); Methylobacterium iners; Methylobacterium populi (Methylorubrum populi); Methylobacterium isbiliense; Methylobacterium pseudosasae (Methylorubrum pseudosasae); Methylobacterium jeotgali; Methylobacterium rhodesianum (Methylorubrum rhodesianum); Methylobacterium komagatae; Methylobacterium rhodinum (Methylorubrum rhodinum); Methylobacterium longum; Methylobacterium salsuginis (Methylorubrum salsuginis); Methylobacterium marchantiae; Methylobacterium suomiense (Methylorubrum suomiense; Methylobacterium mesophilicum; Methylobacterium thiocyanatum (Methylorubrum thiocyanatum); Methylobacterium nodulans; Methylobacterium zatmanii (Methylorubrum zatmanii); or Methylobacterium organophilum.

“Colonization efficiency” as used herein refers to the relative ability of a given microbial strain to colonize a plant host cell or tissue as compared to non-colonizing control samples or other microbial strains. Colonization efficiency can be assessed, for example and without limitation, by determining colonization density, reported for example as colony forming units (CFU) per mg of plant tissue, or by quantification of nucleic acids specific for a strain in a colonization screen, for example using qPCR.

As used herein “mineral nutrients” (also sometime refered to simply as “nutrients”) are micronutrients or macronutrients required or useful for plants or plant parts including for example, but not limited to, nitrogen (N), potassium (K), calcium (Ca), magnesium (Mg), phosphorus (P), and sulfur (S), and the micronutrients chlorine (Cl), Iron (Fe), Boron (B), manganese (Mn), zinc (Z), copper (Cu), molybdenum (Mo) and nickel (Ni).

As used herein, “vitamins” are organic compounds required in small amounts for normal growth and metabolism. Vitamins are important for human and/or animal growth and some vitamins have been reported to be beneficial to plants. Vitamins include but are not limited to vitamin A (including but not limited to all-trans-retinol, all-trans-retinyl-esters, as well as all-trans-beta-carotene and other provitamin A carotenoids), vitamin B1 (thiamine), vitamin B2 (riboflavin), vitamin B3 (niacin), vitamin B5 (pantothenic acid), vitamin B6 (pyridoxine), vitamin B7 (biotin), vitamin B9 (folic acid or folate), vitamin B12 (cobalamins), vitamin C (ascorbic acid), vitamin D (calciferols), vitamin E (tocopherols and tocotrienols), and vitamin K (quinones).

As used herein, the term “strain” shall include all isolates of such strain.

As used herein, “variant” when used in the context of a Methylobacterium isolate, refers to any isolate that has chromosomal genomic DNA with at least 99%, 99.9, 99.8, 99.7, 99.6%, or 99.5% sequence identity to chromosomal genomic DNA of a reference Methylobacterium isolate, such as, for example, a deposited Methylobacterium isolate provided herein. A variant of an isolate can be obtained from various sources including soil, plants or plant material, and water, particularly water associated with plants and/or agriculture. Variants include derivatives obtained from deposited isolates. Methylobacterium isolates or strains can be sequenced (for example as taught by Sanger et al. (1977), Bentley et al. (2008) or Caporaso et al. (2012)) and genome-scale comparison of the sequences conducted (Konstantinidis et al. (2005)) using sequence analysis tools, such as BLAST, as taught by Altschul et al. (1990) or clustalw (www.ebi.ac.uk/Tools/msa/clustalw2/).

As used herein, “derivative” when used in the context of a Methylobacterium isolate, refers to any Methylobacterium that is obtained from a deposited Methylobacterium isolate provided herein. Derivatives of a Methylobacterium isolate include, but are not limited to, derivatives obtained by selection, derivatives selected by mutagenesis and selection, and genetically transformed Methylobacterium obtained from a Methylobacterium isolate. A “derivative” can be identified, for example based on genetic identity to the strain or isolate from which it was obtained and will generally exhibit chromosomal genomic DNA with at least 99%, 99.9, 99.8, 99.7, 99.6%, or 99.5% sequence identity to chromosomal genomic DNA of the strain or isolate from which it was derived.

As used herein, “sequence identity” when used to evaluate whether a particular Methylobacterium strain is a variant or derivative of a Methylobacterium strain provided herein refers to a measure of nucleotide-level genomic similarity between the coding regions of two genomes. Sequence identity between the coding regions of bacterial genomes can be calculated, for example, by determining the Average Nucleotide Identity (ANI) score using FastANI (Jain et al. “High throughput ANI analysis of 90K prokaryotic genomes reveals clear species boundaries”, Nat Communications 9, 5114 (2018)) and Han et al. (“ANI tools web: a web tool for fast genome comparison within multiple bacterial strains”; Database, 2016, 1-5).

As used herein, the term “cultivate” means to grow a plant. A cultivated plant can be one grown and raised on a large agricultural scale or on a smaller scale, including for example a single plant.

As used herein, the term “hydroponic”, “hydroponics” or “hydroponically” refers to a method of cultivating plants in the absence of soil.

Where a term is provided in the singular, other embodiments described by the plural of that term are also provided.

To the extent to which any of the preceding definitions is inconsistent with definitions provided in any patent or non-patent reference incorporated herein by reference, any patent or non-patent reference cited herein, or in any patent or non-patent reference found elsewhere, it is understood that the preceding definition will be used herein.

### Further Description

Isolated Methylobacterium strains that enhance early growth of plants, improve propagation/transplant vigor, increase nutrient uptake, improve stand establishment, and/or improve stress tolerance and compositions useful for treatment of plants with such strains are provided herein. In certain embodiments, the Methylobacterium in the composition is selected from the group consisting of LGP2022, LGP2023 and LGP2021. In certain embodiments, the Methylobacterium in the composition is a variant of LGP2022, LGP2023 or LGP2021. In certain embodiments, early plant development is enhanced, for example prior to a plant reaching the two true leaf stage. In certain embodiments, the plants are fruit or vegetable plants. In certain embodiments, the plants are leafy green plants. In certain embodiments, the plants are grown in a greenhouse. In certain embodiments, the plants are grown hydroponically or in an aeroponic plant cultivation system. Also provided is an isolated Methylobacterium strain selected from LGP2022, LGP2023 and LGP2021.

Further provided are methods of improving production of plants including leafy green plants, fruit and vegetable plants, row crops, such as corn, soybean, wheat, barley and such, and specialty crops, including cannabis crops, by treatment with Methylobacterium strains provided herein. In some embodiments, production is improved by enhanced early growth of treated plants or plants grown from treated seeds in comparison to an untreated control plant or in comparison to a control plant grown from an untreated seed. Such enhanced early growth is measured, for example, by an increase in biomass of treated plants, including increased shoot, leaf, root, or whole seedling biomass. Increased early growth can result in various improvements in plant production, including for example increased biomass production or yield of harvested plants, increased and/or more uniform fruit production, faster seed set, earlier maturation, increased rate of leaf growth, increased rate of root growth, increased seed yield, and decreased cycle time in comparison to an untreated control plant or in comparison to a control plant grown from an untreated seed. In certain embodiments, application of Methylobacterium strains as provided herein provides for a 1%, 2%, 3%, 4%, 5%, 6%, 7%, 8%, 9%, 10%, 12%, 15%, 17%, 20%, 30% or 40% increase in any of the aforementioned traits in comparison to an untreated control plant or in comparison to a control plant grown from an untreated seed. In some embodiments, production is enhanced by increased rooting, for example of plant cuttings, where such increased rooting can result in decreased cycling time and/or increased biomass or yield of the treated plants.

Various methods for identifying a Methylobacterium strain that increases the content of at least one mineral nutrient and/or at least one vitamin in a leafy green plant or plant part are also provided herein. In such methods, a leafy green plant seed and/or a leafy green plant seedling is treated with at least a first Methylobacterium strain to obtain a treated seed and/or a treated plant or plant part, for example a plant cutting. Following cultivation of the treated seed, plant or plant part, a plant part is harvested from the cultivated plant and from a control plant grown from an untreated control seed or untreated control plant, or from a plant treated with a second Methylobacterium strain. The levels in the harvested plant part of the treated and control plants of at least one mineral nutrient and/or vitamin are determined, and a Methylobacterium strain is selected that increases the content of at least one mineral nutrient or vitamin in the cultivated plant or a plant part of the cultivated plant in comparison to the content of the at least one mineral nutrient or vitamin in the cultivated control plant or plant part or in comparison to plants treated with other Methylobacterium strains. In some embodiments, a leafy green plant seed is treated. In other embodiments, a leafy green plant seedling or part thereof is treated. In some embodiments, a leafy green plant seeds or seedlings are separately treated with two, three, four or more Methylobacterium strains and levels of one or more mineral nutrients and vitamins are compared for plants or plant parts treated with different strains, and a Methylobacterium strain or strains demonstrating improved levels of one or more mineral nutrients and vitamins is selected. In some embodiments, plants are treated with combinations of Methylobacterium strains and combinations useful for treatment of leafy green plants to increase vitamins and/or nutrients are identified.

In some embodiments, a leafy green plant seed is treated. In certain other embodiments, a plant seedling or part thereof is treated. In some embodiments, a leafy green plant shoot or seedling is treated. In some embodiments, a leafy green plant seedling is treated prior to development of a first true leaf. In some embodiments, the treated leafy green plant is cultivated to the second true leaf stage and harvested to determine levels of at least one mineral nutrient and/or vitamin. In some embodiments, a treated leafy green plant is cultivated to the third or fourth true leaf stage. In some embodiments, the treated leafy green plant is cultivated for 10 to 14 days. In some embodiments, the treated leafy green plant is cultivated for 14 to 28 days. In some embodiments, the treated leafy green plant is cultivated for 28 or more days prior to harvest and analysis of tissue samples to determine levels of mineral nutrients and vitamins. In some embodiments, treated leafy green plant seeds or seedlings are cultivated in a hydroponic system or an aeroponic plant growth system. A hydroponics system can be a water culture system, a nutrient film technique, an ebb and flow system, a drip system, or a wick system. In an aeroponic system, plants are grown in an air or mist environment without the use of soil. In some embodiments, the hydroponic or aeroponic system can be a variation of any of these types or a combination of one or more systems. In some embodiments, a hydroponic or aeroponic system is advantageous over a soil based cultivation system for determining effects of Methylobacterium strains due to the presence of fewer background microorganisms. Various inert substrates can be used to support the plants, seedlings and root systems in hydroponic or aeroponic growth, including but not limited to perlite, rockwool, clay pellets, foam cubes, rock, peat moss, or vermiculite.

In some embodiments, a Methylobacterium strain tested in the disclosed methods to identify a strain that increases the content of at least one mineral nutrient and/or at least one vitamin in a leafy green plant or plant part, is more efficient at colonizing a plant host cell or tissue, as compared to other Methylobacterium strains. Methods for identifying microbial strains having enhanced colonization efficiency are described in WO2020163027 (PCT/US2020/012041), which is incorporated herein by reference in its entirety.

In some embodiments, a Methylobacterium strain that increases the content of at least one mineral nutrient and/or at least one vitamin in a leafy green plant or plant part, also imparts a trait improvement to said leafy green plant selected from increased biomass production, decreased cycle time, increased rate of leaf growth, decreased time to develop two true leaves, increased rate of root growth, and increased seed yield.

Various methods of using Methylobacterium strains to enhance early growth or rooting, to increase the mineral nutrient and/or vitamin content, to improve propagation/transplant vigor, to improve stand establishment, and/or to improve stress tolerance in plants, such as leafy green plants, row crops, cannabis and other speciality crops are provided herein. In certain embodiments, Methylobacterium treatment of a row crop, including but not limited to corn, soybean, rice, canola, and wheat, results in enhanced plant growth and yield. In certain embodiments, the crop is rice and the Methylobacterium is selected from the group consisting of LGP2016 (ISO117), LGP2017 (ISO118), LGP2019 (ISO120) and variants thereof. In some embodiments, Methylobacterium selected from LGP2001, LGP2002, LGP2009, LGP2015, a combination of LGP2002 and LGP2015, and variants thereof is/are applied to rosemary, French tarragon, basil, Pennisetum, and other herbs to improve growth and root development. In certain embodiments, Methylobacterium treatment of soil, a seed, a leaf, a stem, a root, or a shoot can enhance early growth, propagation/transplant vigor, stand establishment, and/or stress tolerance as well as or alternatively increase the content of one or more mineral nutrients or vitamins in harvested leafy green plants or plant parts from plants grown from the Methylobacterium-treated plant parts or Methylobacterium-treated seeds provided herein. In some embodiments, Methylobacterium LGP2022, LGP2023, LGP2021 or variants thereof are applied to plants, plant parts or seeds to enhance early plant growth and improve plant production.

Alternatively, such Methylobacterium may be applied to soil or other growth medium where plants are grown. Methylobacterium soil treatments or applications can include, but are not limited to, in-furrow applications (e.g., before, during, and/or after seed deposition), soil drenches, distribution of granular or other dried formulations to the soil (e.g., before, during, and/or after seed deposition or plant growth). Methylobacterium treatments for plants grown in hydroponic systems can include seed treatments prior to germination, foliar applications to germinated plants or parts thereof, and applications in a liquid solution used in the hydroponic system. In certain embodiments, Methylobacterium treatment of a leafy green plant can include application to the seed, plant, and/or a part of the plant and can thus comprise any Methylobacterium treatment or application resulting in colonization of the leafy green plant by the Methylobacterium. In some embodiments, application of Methylobacterium to crops that are propagated by cutting can enhance growth and/or rooting of such plants. Field transplants of such treated and rooted cuttings may demonstrate decreased cycling time, and/or improved biomass and/or yield as a result of such treatments. In some embodiments, Methylobacterium selected from LGP2002, LGP2009, LGP2019 and variants thereof are applied to cannabis cuttings to improve growth and root development.

Treatments or applications to plants described herein can include, but are not limited to, spraying, coating, partially coating, immersing, and/or imbibing the seed, plant or plant parts with the Methylobacterium strains and compositions comprising the same provided herein. In certain embodiments, soil, a seed, a leaf, a stem, a root, a tuber, or a shoot can be sprayed, immersed and/or imbibed with a liquid, semi-liquid, emulsion, or slurry of a composition provided herein. Such treatments, applications, seed immersion, or imbibition can be sufficient to provide for enhanced early growth and/or increased levels of one or more mineral nutrients and/or vitamins content in harvestable tissue from a treated plant or plant grown from a treated seed in comparison to an untreated plant or plant grown from an untreated seed. Enhanced early growth can lead to further improvements in plant production including an increase in biomass of treated plants, such as increased shoot, root, or whole seedling biomass. Enhanced early growth can result in various additional improvements in plant production, including for example increased yield of harvested plants or harvested plant parts, increased and/or more uniform fruit production, faster seed set, earlier maturation, increased rate of leaf growth, increased rate of root growth, increased seed yield, and decreased cycle time. In certain embodiments, plant seeds or cuttings can be immersed and/or imbibed for at least 1, 2, 3, 4, 5, or 6 hours. Such immersion and/or imbibition can, in certain embodiments, be conducted at temperatures that are not deleterious to the plant seed or the Methylobacterium. In certain embodiments, the seeds can be treated at about 15 to about 30 degrees Centigrade or at about 20 to about 25 degrees Centigrade. In certain embodiments, seed imbibition and/or immersion can be performed with gentle agitation. Seed treatments can be effected with both continuous and/or batch seed treaters. In certain embodiments, the coated seeds can be prepared by slurrying seeds with a coating composition comprising a Methylobacterium strain that increases the levels of one or more mineral nutrients and/or vitamins and air-drying the resulting product. Air-drying can be accomplished at any temperature that is not deleterious to the seed or the Methylobacterium, but will typically not be greater than 30 degrees Centigrade. The proportion of coating that comprises the Methylobacterium strain includes, but is not limited to, a range of 0.1 to 25% by weight of the seed or other plant part, 0.5 to 5% by weight of the seed or other plant part, and 0.5 to 2.5% by weight of the seed or other plant part. In certain embodiments, a solid substance used in the seed coating or treatment will have a Methylobacterium strain that increases mineral nutrient and or vitamin content adhered to a solid substance as a result of being grown in biphasic media comprising the Methylobacterium strain, solid substance, and liquid media. Methods for growing Methylobacterium in biphasic media include those described in U.S. Pat. No. 9,181,541, which is specifically incorporated herein by reference in its entirety. In certain embodiments, compositions suitable for treatment of a seed or plant part can be obtained by the methods provided in U.S. Pat. No. US 10,287,544, which is specifically incorporated herein by reference in its entirety. Various seed treatment compositions and methods for seed treatment disclosed in U.S. Pat. Nos. 5,106,648, 5,512,069, and 8,181,388 are incorporated herein by reference in their entireties and can be adapted for treating seeds with compositions comprising a Methylobacterium strain.

In certain embodiments where plant seeds are treated with Methylobacterium compositions provided herein, the compositions further comprise one or more lubricants to ensure smooth flow and separation (singulation) of seeds in the seeding mechanism, for example a planter box. Lubricants for use in such compositions include talc, graphite, polyethylene wax based powders (such as Fluency Agent), protein powders, for example soybean protein powders, or a combination of protein powders and a lipid, for example lecithin or a vegetable oil. Lubricants can be applied to seeds simultaneously with application of Methylobacterium, or may be mixed with Methylobacterium prior to application of the compositions to the seeds.

In certain embodiments, treated plants are cultivated in a hydroponic system. In some embodiments, plant seeds are treated and plants are grown from the treated seeds continuously in the same cultivation system. In some embodiments, plant seeds are treated and cultivated in a hydroponic nursery to produce seedlings. The seedlings transferred to a different hydroponic system for commercial production of leafy greens. In some embodiments, a Methylobacterium strain that enhances early growth or increases the levels of one or more mineral nutrients and/or vitamins persists in the seedlings transferred to a greenhouse production system and continues to provide advantages such as improved micronutrient and/or vitamin content and/or biomass production, through the further growth of the leafy green plant. In some embodiments, plant seedlings transferred to a greenhouse production system may be further treated with LGP2009, LGP2022, LGP2023, LGP2021 or variants thereof, or with one or more other Methylobacterium strains that increase the levels of one or more mineral nutrients and/or vitamins prior to, during or after transfer to the production system.

In certain embodiments, the composition used to treat the seed or plant part can contain a Methylobacterium strain and an agriculturally acceptable excipient. Agriculturally acceptable excipients include, but are not limited to, woodflours, clays, activated carbon, diatomaceous earth, fine-grain inorganic solids, calcium carbonate and the like. Clays and inorganic solids that can be used with the include, but are not limited to, calcium bentonite, kaolin, china clay, talc, perlite, mica, vermiculite, silicas, quartz powder, montmorillonite and mixtures thereof. Agriculturally acceptable excipients also include various lubricants such as talc, graphite, polyethylene wax based powders (such as Fluency Agent), protein powders, for example soybean protein powders, or a combination of protein powders and a lipid, for example lecithin or a vegetable oil.

Agriculturally acceptable adjuvants that promote sticking to the seed that can be used include, but are not limited to, polyvinyl acetates, polyvinyl acetate copolymers, hydrolyzed polyvinyl acetates, polyvinylpyrrolidone-vinyl acetate copolymer, polyvinyl alcohols, polyvinyl alcohol copolymers, polyvinyl methyl ether, polyvinyl methyl ether-maleic anhydride copolymer, waxes, latex polymers, celluloses including ethylcelluloses and methylcelluloses, hydroxy methylcelluloses, hydroxypropylcellulose, hydroxymethylpropylcelluloses, polyvinyl pyrrolidones, alginates, dextrins, malto-dextrins, polysaccharides, fats, oils, proteins, karaya gum, jaguar gum, tragacanth gum, polysaccharide gums, mucilage, gum arabics, shellacs, vinylidene chloride polymers and copolymers, soybean-based protein polymers and copolymers, lignosulfonates, acrylic copolymers, starches, polyvinylacrylates, zeins, gelatin, carboxymethylcellulose, chitosan, polyethylene oxide, acrylamide polymers and copolymers, polyhydroxyethyl acrylate, methylacrylamide monomers, alginate, ethylcellulose, polychloroprene and syrups or mixtures thereof. Other useful agriculturally acceptable adjuvants that can promote coating include, but are not limited to, polymers and copolymers of vinyl acetate, polyvinylpyrrolidone-vinyl acetate copolymer and water-soluble waxes. Further, agriculturally acceptable adjuvants also include various lubricants (wich can provide for smooth flow and separation (singulation) of seeds) such as talc, graphite, polyethylene wax based powders (such as Fluency Agent), protein powders, for example soybean protein powders, or a combination of protein powders and a lipid, for example lecithin or a vegetable oil. Various surfactants, dispersants, anticaking-agents, foam-control agents, and dyes disclosed herein and in U.S. Pat. No. 8,181,388 can be adapted for use with compositions comprising a suitable Methylobacterium strain. In certain embodiments, the seed and/or seedling is exposed to the composition by providing the Methylobacterium strain in soil in which the plant or a plant arising from the seed are grown, or other plant growth media in which the plant or a plant arising from the seed are grown. Examples of methods where the Methylobacterium strain is provided in the soil include in furrow applications, soil drenches, and the like.

Non-limiting examples of treatments of plant seeds, seedling or other plant parts with a Methylobacterium providing for enhanced early growth and/or increased content of one or more mineral nutrients and/or vitamins in a harvested plant part include treatments of vegetable crops with edible leaves including, without limitation, spinach, kale, lettuce (including but not limited to romaine, butterhead, iceberg and loose leaf lettuces), field greens, including brassica greens. Specific greens that can be treated with Methylobacterium provided herein include collard greens, cabbage, beet greens, watercress, swiss chard, arugula, escarole, endive, bok choy and turnip greens. Other leafy green plants that are grown for production and harvest of microgreens and/or herbs, can also be treated in the methods described herein to provide for increased content of one or more mineral nutrients and/or vitamins in harvested microgreens and herbs, including but not limited to lettuce, cauliflower, broccoli, cabbage, watercress, arugula, garlic, onion, leek, amaranth, swill chard, been, spinach, melon, cucumber, squash, basil, celery, cilantro, radish, radicchio, chicory, dill, rosemary, French tarragon, basil, Pennisetum, carrot, fennel, beans, peas, chickpeas, and lentils. Treatment of plants grown for harvest of fleshy fruits are also provided herein. Such plants include, for example, melon (including watermelon and cantaloupe), berry (including strawberry, blueberry, blackberry and raspberry), grape, kiwi, mango, papaya, pineapple, banana, pepper, tomato, squash, and cucumber plants.

In certain embodiments, LGP2022, LGP2023, LGP2021 or variants thereof will also find use in treatment of other plant species to enhance early growth, including, for example field crops, ornamentals, turf grasses and trees grown in commercial production, such as conifer trees. Without limitation, such additional plant species include corn, soybean, cruciferous or Brassica sp. vegetables (e.g., B. napus, B. rapa, B. juncea), alfalfa, rice, rye, wheat, barley, oats, sorghum, millet (e.g., pearl millet (Pennisetum glaucum), proso millet (Panicum miliaceum), foxtail millet (Setaria italica), and finger millet (Eleusine coracana)), sunflower, safflower, tobacco, potato, peanuts, cotton, species in the genus Cannabis (including, but not limited to, Cannabis sativa and industrial hemp varieties), sweet potato (Ipomoea batatus), cassava, coffee, coconut, ornamentals (including, but not limited to, azalea, hydrangea, hibiscus, roses, tulips, daffodils, petunias, carnation, poinsettia, and chrysanthemum), conifers (including, but not limited to pines such as loblolly pine, slash pine, ponderosa pine, lodge pole pine, and Monterey pine; Douglas-fir; Western hemlock; Sitka spruce; redwood; true firs such as silver fir and balsam fir; and cedars such as Western red cedar and Alaska yellow-cedar) and turfgrass (including, but are not limited to, annual bluegrass, annual ryegrass, Canada bluegrass, fescue, bentgrass, wheatgrass, Kentucky bluegrass, orchard grass, ryegrass, redtop, Bermuda grass, St. Augustine grass, and zoysia grass).

In certain embodiments, a Methylobacterium strain used to treat a given cultivar or variety of plant seed, plant or plant part can be a Methylobacterium strain that was isolated from a different plant species, or a different cultivar or variety of the plant species being treated, and is thus heterologous or non-resident to the treated plant or plant part. Plant parts that have increased levels of one or more mineral nutrients and/or vitamins as the result of treatment with Methylobacterium as provided herein include, but are not limited to, leaves, stems, flowers, roots, seeds, fruit, tubers, coleoptiles, and the like. In certain embodiments, a plant having enhanced early growth as a result of treatment with LGP2022, LGP2023, LGP2021 or variants thereof, or a plant having enhanced levels of one or more mineral nutrients as a results of treatment with Methylobacterium compositions provided herein is a leafy green plant. In some embodiments, increased levels of one or more mineral nutrients and/or vitamins are present in a leaf. In certain embodiments, the increased levels of one or more mineral nutrients and/or vitamins are present in the harvested greens, including leaves and shoots.

In certain embodiments, a manufactured combination composition comprising two or more Methylobacterium strains can be used to treat a seed or plant part in any of the methods provided herein. Such manufactured combination compositions can be made by methods that include harvesting monocultures of each Methylobacterium strain and mixing the harvested monocultures to obtain the manufactured combination composition of Methylobacterium. In certain embodiments, the manufactured combination composition of Methylobacterium can comprise Methylobacterium isolated from different plant species or from different cultivars or varieties of a given plant.

In certain embodiments, an effective amount of the Methylobacterium strain or strains used in treatment of plants, seeds or plant parts is a composition having a Methylobacterium titer of at least about 1×10⁶ colony-forming units per milliliter, at least about 5×10⁶ colony-forming units per milliliter, at least about 1×10⁷ colony-forming units per milliliter, at least about 5 × 10⁸ colony-forming units per milliliter, at least about 1 × 10⁹ colony-forming units per milliliter, at least about 1 × 10¹⁰ colony-forming units per milliliter, or at least about 3 × 10¹⁰ colony-forming units per milliliter. In certain embodiments, an effective amount of the Methylobacterium strain or strains is a composition with the Methylobacterium at a titer of about least about 1×10⁶ colony-forming units per milliliter, at least about 5×10⁶ colony-forming units per milliliter, at least about 1×10⁷ colony-forming units per milliliter, or at least about 5 × 10⁸ colony-forming units per milliliter to at least about 6 × 10¹⁰ colony-forming units per milliliter of a liquid or an emulsion. In certain embodiments, an effective amount of the Methylobacterium strain or strains is a composition with the Methylobacterium at least about 1×10⁶ colony-forming units per gram, at least about 5×10⁶ colony-forming units per gram, at least about 1×10⁷ colony-forming units per gram, or at least about 5 × 10⁸ colony-forming units per gram to at least about 6 × 10¹⁰ colony-forming units of Methylobacterium per gram of the composition. In certain embodiments, an effective amount of a composition provided herein can be a composition with a Methylobacterium titer of at least about 1×10⁶ colony-forming units per gram, at least about 5×10⁶ colony-forming units per gram, at least about 1×10⁷ colony-forming units per gram, or at least about 5×10⁸ colony-forming units per gram to at least about 6×10¹⁰ colony-forming units of Methylobacterium per gram of particles in the composition containing the particles that comprise a solid substance wherein a mono-culture or co-culture of Methylobacterium strain or strains is adhered thereto. In certain embodiments, an effective amount of a composition provided herein to a plant or plant part can be a composition with a Methylobacterium titer of at least about 1×10⁶ colony-forming units per mL, at least about 5×10⁶ colony-forming units per mL, at least about 1×10⁷ colony-forming units per mL, or at least about 5 × 10⁸ colony-forming units per mL to at least about 6 × 10¹⁰ colony-forming units of Methylobacterium per mL in a composition comprising an emulsion wherein a mono-culture or co-culture of a Methylobacterium strain or strains adhered to a solid substance is provided therein or grown therein. In certain embodiments, an effective amount of a composition provided herein can be a composition with a Methylobacterium titer of at least about 1×10⁶ colony-forming units per mL, at least about 5×10⁶ colony-forming units per mL, at least about 1×10⁷ colony-forming units per mL, or at least about 5 × 10⁸ colony-forming units per mL to at least about 6 × 10¹⁰ colony-forming units of Methylobacterium per mL in a composition comprising an emulsion wherein a mono-culture or co-culture of a Methylobacterium strain or strains is provided therein or grown therein. In certain embodiments, any of the aforementioned compositions comprising a mono-culture or co-culture of a Methylobacterium strain or strains can further comprise a mono- or co-culture of Rhizobium and/or Bradyrhizobium.

In certain embodiments, an effective amount of a Methylobacterium strain or strains that provides for increased early growth and/or increased mineral nutrient and/or vitamin content provided in a treatment of a seed or plant part is at least about 10³, 10⁴, 10⁵, or 10⁶ CFU per seed or treated plant part. In certain embodiments, an effective amount of Methylobacterium provided in a treatment of a seed or plant part is at least about 10³, 10⁴, 10⁵, or 10⁶ CFU to about 10⁷, 10⁸, 10⁹, or 10¹⁰ CFU per seed or treated plant part. In certain embodiments, the effective amount of Methylobacterium provided in a treatment of a seed or plant part is an amount where the CFU per seed or treated plant part will exceed the number of CFU of any resident naturally occurring Methylobacterium strain by at least 5-, 10-, 100-, or 1000-fold. In certain embodiments, the effective amount of Methylobacterium provided in a treatment of a seed or plant part is an amount where the CFU per seed or treated plant part will exceed the number of CFU of any resident naturally occurring Methylobacterium by at least 2-, 3-, 5-, 8-, 10-, 20-, 50-, 100-, or 1000-fold. In certain embodiments where the treated plant is cultivated in a hydroponic system, populations of naturally occurring Methylobacterium or other soil microbes will be minimal.

Non-limiting examples of Methylobacterium strains that can be used in methods provided herein are disclosed in Table 1. Other Methylobacterium strains useful in certain methods provided herein include variants of the Methylobacterium strains disclosed in Table 1. Also of use are various combinations of two or more strains or variants of Methylobacterium strains disclosed in Table 1 for treatment of plants or parts thereof.

TABLE 1

Methylobacterium sp. strain

| | | | | |
|---|---|---|---|---|
| Deposit Identifier | Isolate No. | LGP NO. | USDA ARS NRRL No.¹ | Strain Source: Obtained from: |
| Methylobacterium sp. #1 | ISO101 | LGP2000 | NRRL B-50929 | a soybean plant grown in Saint Louis County, Missouri, USA |
| Methylobacterium sp. #2 | ISO102 | LGP2001 | NRRL B-50930 | a weed grown in Saint Louis County, Missouri, USA |
| Methylobacterium sp. #3 | ISO103 | LGP2002 | NRRL B-50931 | a mint plant grown in Saint Louis County, Missouri, USA |
| Methylobacterium sp. #4 | ISO104 | LGP2003 | NRRL B-50932 | a soybean plant grown in Saint Louis County, Missouri, USA |
| Methylobacterium sp. #5 | ISO105 | LGP2004 | NRRL B-50933 | a broccoli plant grown in Saint Louis County, Missouri, USA |
| Methylobacterium sp. #6 | ISO106 | LGP2005 | NRRL B-50934 | a corn plant grown in Saint Louis County, Missouri, USA |
| Methylobacterium sp. #7 | ISO107 | LGP2006 | NRRL B-50935 | a corn plant grown in Saint Louis County, Missouri, USA |
| Methylobacterium sp. #8 | ISO108 | LGP2007 | NRRL B-50936 | a corn plant grown in Saint Louis County, Missouri, USA |
| Methylobacterium sp. #9 | ISO109 | LGP2008 | NRRL B-50937 | a corn plant grown in Saint Louis County, Missouri, USA |
| Methylobacterium sp. #10 | ISO110 | LGP2009 | NRRL B-50938 | a corn plant grown in Saint Louis County, Missouri, USA |
| Methylobacterium sp. #11 | ISO111 | LGP2010 | NRRL B-50939 | a lettuce plant grown in Saint Louis County, Missouri, USA |
| Methylobacterium sp. #12 | ISO112 | LGP2011 | NRRL B-50940 | a corn plant grown in Saint Louis County, Missouri, USA |
| Methylobacterium sp. #13 | ISO113 | LGP2012 | NRRL B-50941 | a tomato plant grown in Saint Louis County, Missouri, USA |
| Methylobacterium sp. #14 | ISO114 | LGP2013 | NRRL B-50942 | a tomato plant grown in Saint Louis County, Missouri, USA |
| Methylobacterium sp. #15 | ISO115 | LGP2014 | NRRL B-67339 | a soybean plant grown in Saint Louis County, Missouri, USA |
| Methylobacterium sp. #16 | ISO116 | LGP2015 | NRRL B-67340 | a yucca plant grown in Saint Louis County, Missouri, USA |
| Methylobacterium sp. #17 | ISO117 | LGP2016 | NRRL B-67341 | a soybean plant grown in Saint Louis County, Missouri, USA |
| Methylobacterium sp. #18 | ISO118 | LGP2017 | NRRL B-67741 | a Dionaea muscipula plant (Venus fly trap) grown in St. Charles, MO. |
| Methylobacterium sp. #19 | ISO119 | LGP2018 | NRRL B-67742 | an Orchidaceae spp. plant (orchid) grown in Saint Louis County, Missouri, USA |
| Methylobacterium sp. #20 | ISO120 | LGP2019 | NRRL B-67743 | a tomato plant grown in Saint Louis County, Missouri, USA |
| Methylobacterium sp. #26 | ISO121 | LGP2020 | NRRL-B-67892 | A Lagerstroemia indica (crape myrtle) plant grown in Saint Louis County, Missouri, USA |
| Methylobacterium sp. #28 | | LGP2021 | NRRL-B-68032 | A Cichorium intybus (chicory) plant growing in Saint Louis County, Missouri, USA |
| Methylobacterium sp. #29 | | LGP2022 | NRRL-B-68033 | A Coronilla vario (crown vetch) plant growing in Saint Louis County, Missouri, USA |
| Methylobacterium sp. #30 | | LGP2023 | NRRL-B-68034 | A Catharanthus roseus (periwinkle) growing in Fort Myers, Florida, USA |

¹ Deposit number for strain deposited with the AGRICULTURAL RESEARCH SERVICE CULTURE COLLECTION (NRRL) of the National Center for Agricultural Utilization Research, Agricultural Research Service, U.S. Department of Agriculture, 1815 North University Street, Peoria, Illinois 61604 U.S.A. under the terms of the Budapest Treaty on the International Recognition of the Deposit of Microorganisms for the Purposes of Patent Procedure. Subject to 37 CFR §1.808(b), all restrictions imposed by the depositor on the availability to the public of the deposited material will be irrevocably removed upon the granting of any patent from this patent application.

Variants of a Methylobacterium isolate listed in Table 1 include isolates obtained therefrom by genetic transformation, mutagenesis and/or insertion of a heterologous sequence. In some embodiments, such variants are identified by the presence of chromosomal genomic DNA with at least 99%, 99.9, 99.8, 99.7, 99.6%, or 99.5% sequence identity to chromosomal genomic DNA of the strain from which it was derived. In certain embodiments, such variants are distinguished by the presence of one or more unique DNA sequences that include: (i) a unique sequence of SEQ ID NOs: 1 to 3, SEQ ID NOs: 13 to 15, SEQ ID NOs: 25 to 27, SEQ ID NOs: 37 to 39, SEQ ID NOs: 49 to 51, and SEQ ID NOs: 61 to 73; or (ii) sequences with at least 98% or 99% sequence identity across the full length of SEQ ID NOs: 1 to 3, SEQ ID NOs: 13 to 15, SEQ ID NOs: 25 to 27, SEQ ID NOs: 37 to 39, SEQ ID NOs: 49 to 51, SEQ ID NOs: 61 to 73, and SEQ ID Nos:74 to 76.

In certain embodiments of the methods provided herein, the Methylobacterium strain or strains used to treat a leafy green plant seed and/or a plant part are selected from the group consisting of ISO101 (NRRL B-50929), ISO102 (NRRL B-50930), ISO103 (NRRL B-50931), ISO104 (NRRL B-50932), ISO105 (NRRL B-50933), ISO106 (NRRL B-50934), ISO107 (NRRL B-50935), ISO108 (NRRL B-50936), ISO109 (NRRL B-50937), ISO110 (NRRL B-50938), ISO111 (NRRL B-50939), ISO112 (NRRL B-50940), ISO113 (NRRL B-50941), ISO114 (NRRL B-50942), ISO115 (NRRL B-67339), ISO116 (NRRL B-67340), ISO117 (NRRL B-67341), ISO118 (NRRL B-67741), ISO119 (NRRL B-67742), ISO120 (NRRL B-67743), ISO121 (NRRL-B-67892), variants thereof, or any combination thereof. In certain embodiments, one or more of the Methylobacterium strains used in the methods can comprise total genomic DNA (chromosomal and plasmid DNA) or average nucleotide identity (ANI) with at least 99%, 99.9, 99.8, 99.7, 99.6%, or 99.5% sequence identity or ANI to total genomic DNA of ISO101 (NRRL B-50929), ISO102 (NRRL B-50930), ISO103 (NRRL B-50931), ISO104 (NRRL B-50932), ISO105 (NRRL B-50933), ISO106 (NRRL B-50934), ISO107 (NRRL B-50935), ISO108 (NRRL B-50936), ISO109 (NRRL B-50937), ISO110 (NRRL B-50938), ISO111 (NRRL B-50939), ISO112 (NRRL B-50940), ISO113 (NRRL B-50941), ISO114 (NRRL B-50942), ISO115 (NRRL B-67339), ISO116 (NRRL B-67340), ISO117 (NRRL B-67341), ISO118(NRRL B-67741), ISO119 (NRRL B-67742), ISO120 (NRRL B-67743) or ISO121 (NRRL-B-67892). In certain embodiments, the percent ANI can be determined as disclosed by Konstantinidis et al., 2006. In certain embodiments of the methods provided herein, the Methylobacterium strain or strains used to treat a seed and/or a plant part is ISO110 or LGP2009 which was deposited under the NRRL accession No. NRRL B-50938. In certain embodiments, the strain identified as either ISO110 or LGP2009 which was deposited under the NRRL accession No. NRRL B-50938 is used as a control or reference standard for comparison to one or more new test or candidate Methylobacterium isolates in a method of identifying a new Methylobacterium that can improve levels of one or more mineral nutrients and/or vitamins in a leafy greens harvested from a treated plant.

In certain embodiments of the methods provided herein, plants, plant seeds and/or plant parts are treated with both a Methylobacterium strain and at least one additional component. In some embodiments an additional component can be an additional active ingredient, for example, a pesticide or a second biological. In certain embodiments, the pesticide can be an insecticide, a fungicide, an herbicide, a nematicide or other biocide. The second biological could be a strain that improves yield or controls an insect, pest, fungi, weed, or nematode. In some embodiments, a second biological is a second Methylobacterium strain.

Non-limiting examples of insecticides and nematicides include carbamates, diamides, macrocyclic lactones, neonicotinoids, organophosphates, phenylpyrazoles, pyrethrins, spinosyns, synthetic pyrethroids, tetronic and tetramic acids. In particular embodiments insecticides and nematicides include abamectin, aldicarb, aldoxycarb, bifenthrin, carbofuran, chlorantraniliporle, chlothianidin, cyfluthrin, cyhalothrin, cypermethrin, deltamethrin, dinotefuran, emamectin, ethiprole, fenamiphos, fipronil, flubendiamide, fosthiazate, imidacloprid, ivermectin, lambda-cyhalothrin, milbemectin, nitenpyram, oxamyl, permethrin, tioxazafen, spinetoram, spinosad, spirodichlofen, spirotetramat, tefluthrin, thiacloprid, thiamethoxam, and thiodicarb.

Non-limiting examples of useful fungicides include aromatic hydrocarbons, benzimidazoles, benzthiadiazole, carboxamides, carboxylic acid amides, morpholines, phenylamides, phosphonates, quinone outside inhibitors (e.g. strobilurins), thiazolidines, thiophanates, thiophene carboxamides, and triazoles. Particular examples of fungicides include acibenzolar-S-methyl, azoxystrobin, benalaxyl, bixafen, boscalid, carbendazim, cyproconazole, dimethomorph, epoxiconazole, fluopyram, fluoxastrobin, flutianil, flutolanil, fluxapyroxad, fosetyl-Al, ipconazole, isopyrazam, kresoxim-methyl, mefenoxam, metalaxyl, metconazole, myclobutanil, orysastrobin, penflufen, penthiopyrad, picoxystrobin, propiconazole, prothioconazole, pyraclostrobin, sedaxane, silthiofam, tebuconazole, thifluzamide, thiophanate, tolclofos-methyl, trifloxystrobin, and triticonazole. Non-limiting examples of other biocides, include isothiazolinones, for example 1,2 Benzothiazolin-3-one (BIT), 5-Chloro-2-methyl-4-isothiazolin-3-one (CIT), 2-Methyl-4-isothiazolin-3-one (MIT), octylisothiazolinone (OIT), dichlorooctylisothiazolinone (DCOIT), and butylbenzisothiazolinone (BBIT); 2-Bromo-2-nitropropane-1,3-diol (Bronopol), 5-bromo-5-nitro-1,3-dioxane (Bronidox), Tris(hydroxymethyl)nitromethane, 2,2-Dibromo-3-nitrilopropionamide (DBNPA), and alkyl dimethyl benzyl ammonium chlorides.

Non-limiting examples of herbicides include ACCase inhibitors, acetanilides, AHAS inhibitors, carotenoid biosynthesis inhibitors, EPSPS inhibitors, glutamine synthetase inhibitors, PPO inhibitors, PS II inhibitors, and synthetic auxins, Particular examples of herbicides include acetochlor, clethodim, dicamba, flumioxazin, fomesafen, glyphosate, glufosinate, mesotrione, quizalofop, saflufenacil, sulcotrione, and 2,4-D.

In some embodiments, the composition or method disclosed herein may comprise a Methylobacterium strain and an additional active ingredient selected from the group consisting of clothianidin, ipconazole, imidacloprid, metalaxyl, mefenoxam, tioxazafen, azoxystrobin, thiomethoxam, fluopyram, prothioconazole, pyraclostrobin, and sedaxane.

In some embodiments, the composition or method disclosed herein may comprise an additional active ingredient, which may be a second biological. The second biological could be a biological control agent, other beneficial microorganisms, microbial extracts, natural products, plant growth activators or plant defense agent. Non-limiting examples of the second biological could include bacteria, fungi, beneficial nematodes, and viruses. In certain embodiments, the second biological can be a Methylobacterium. In certain embodiments, the second biological is a Methylobacterium listed in Table 1. In certain embodiments, the second biological can be a Methylobacterium selected from M. gregans, M. radiotolerans, M. extorquens, M. populi, M. salsuginis, M. brachiatum, and M. komagatae.

In certain embodiments, the second biological can be a bacterium of the genus Actinomycetes, Agrobacterium, Arthrobacter, Alcaligenes, Aureobacterium, Azobacter, Azorhizobium, Azospirillum, Azotobacter, Beijerinckia, Bacillus, Brevibacillus, Burkholderia, Chromobacterium, Clostridium, Clavibacter, Comomonas, Corynebacterium, Curtobacterium, Enterobacter, Flavobacterium, Gluconacetobacter, Gluconobacter, Herbaspirillum, Hydrogenophage, Klebsiella, Luteibacter, Lysinibacillus, Mesorhizobium, Methylobacterium, Microbacterium, Ochrobactrum, Paenibacillus, Pantoea, Pasteuria, Phingobacterium, Photorhabdus, Phyllobacterium, Pseudomonas, Rhizobium, Rhodococcus, Bradyrhizobium, Serratia, Sinorhizobium, Sphingomonas, Streptomyces, Stenotrophomonas, Variovorax, Xanthomonas and Xenorhadbus. In particular embodiments, the bacteria is selected from the group consisting of Bacillus amyloliquefaciens, Bacillus cereus, Bacillus firmus, Bacillus, lichenformis, Bacillus pumilus, Bacillus sphaericus, Bacillus subtilis, Bacillus thuringiensis, Chromobacterium suttsuga, Pasteuria penetrans, Pasteuria usage, and Pseudomona fluorescens.

In certain embodiments the second biological can be a fungus of the genus Acremonium, Alternaria, Ampelomyces, Aspergillus, Aureobasidium, Beauveria, Botryosphaeria, Cladosporium, Cochliobolus, Colletotrichum, Coniothyrium, Embellisia, Epicoccum, Fusarium, Gigaspora, Gliocladium, Glomus, Laccaria, Metarhisium, Muscodor, Nigrospora, Paecilonyces, Paraglomus, Penicillium, Phoma, Pisolithus, Podospora, Rhizopogon, Scleroderma, Trichoderma, Typhula, Ulocladium, and Verticilium. In particular embodiments, the fungus is Beauveria bassiana, Coniothyrium minitans, Gliocladium vixens, Muscodor albus, Paecilomyces lilacinus, or Trichoderma polysporum.

In further embodiments the second biological can be plant growth activators or plant defense agents including, but not limited to harpin, Reynoutria sachalinensis, jasmonate, lipochitooligosaccharides, and isoflavones.

In further embodiments, the second biological can include, but are not limited to, various Bacillus sp., Pseudomonas sp., Coniothyrium sp., Pantoea sp., Streptomyces sp., and Trichoderma sp. Microbial biopesticides can be a bacterium, fungus, virus, or protozoan. Particularly useful biopesticidal microorganisms include various Bacillus subtilis, Bacillus thuringiensis, Bacillus pumilis, Pseudomonas syringae, Trichoderma harzianum, Trichoderma virens, and Streptomyces lydicus strains. Other microorganisms that are added can be genetically engineered or wild-type isolates that are available as pure cultures. In certain embodiments, it is anticipated that the second biological can be provided in the composition in the form of a spore.

Leafy green plants or harvested plant parts having increased levels of at least one mineral nutrient and/or at least one vitamin in comparison to a control plant, or plant part are provided, as are methods for obtaining and using such plants and plant parts. In certain embodiments, the content of at least one mineral nutrient and/or at least one vitamin in the plants or harvested plant part is increased by at least about 1%, or 2% to about 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, or 30% per gram dry or wet weight in comparison to the content of the at least one mineral nutrient and/or at least one vitamin in a control plant or plant part. In other embodiments, the content of at least one mineral nutrient and/or at least one vitamin in the plants, plant parts, food ingredients, and feed ingredients is increased by more than 30%, including 35%, 40%, 45%, 50% or greater than 50% in comparison to the content of the at least one mineral nutrient and/or at least one vitamin in a control plant or plant part. In some embodiments, the content of more than one mineral nutrient and/or more than one vitamin is increased in a leafy green plant or harvested plant part, and percent increases can vary for each of the mineral nutrients and/or vitamins, with each increased mineral nutrient and vitamin being increased by at least about 1%, or 2% to about 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, or 30% or more per gram dry or wet weight. Controls include plants or plant parts harvested from control plants grown from an untreated control seed or untreated control.

The mineral nutrient and/or content of leafy green plants or harvested parts thereof grown from seeds or seedlings treated with an effective amount of a Methylobacterium strain or strains can be determined by a variety of different techniques or combinations of techniques. Nitrate and nitrite nitrogen content determination methods include Cadmium Reduction and Colorimetric analysis by Flow Injection system (Lachat); AOAC 968.07. Mineral Digestion can be accomplished by Open Vessel Microwave SW846-3051A (AOAC 991-10D(e)). Mineral analysis can be conducted by Inductively Coupled Argon Plasma (ICAP); AOAC 985.01. Mineral nutrients and vitamins content of seeds and various food products can also be determined by standard methods set forth by the AACC, AOAC in Official Methods of Analysis of AOAC INTERNATIONAL, 21st Edition (2019) and in the Codex Alimentarius of International Food Standards set forth by the Food and Agriculture Organization of the United Nations (FAO) or WHO (CXS 234-19991, Adopted in 1999).

### Deposit Information

Samples of the following Methylobacterium sp. strains have been deposited with the AGRICULTURAL RESEARCH SERVICE CULTURE COLLECTION (NRRL) of the National Center for Agricultural Utilization Research, Agricultural Research Service, U.S. Department of Agriculture, 1815 North University Street, Peoria, Illinois 61604 U.S.A. under the terms of the Budapest Treaty on the International Recognition of the Deposit of Microorganisms for the Purposes of Patent Procedure. Methylobacterium sp. NRRL B-50929, NRRL B-50930, NRRL B-50931, NRRL B-50932, NRRL B-50933, NRRL B-50934, NRRL B-50935, NRRL B-50936, NRRL B-50937, NRRL B-50938, NRRL B-50939, NRRL B-50940, NRRL B-50941 and NRRL B-50942 were deposited with NRRL on Mar. 12, 2014. Methylobacterium sp. NRRL B-67339 was deposited with NRRL on Nov. 18, 2016. Methylobacterium sp. NRRL B-67340 was deposited with NRRL on Nov. 18, 2016. Methylobacterium sp. NRRL B-67341 was deposited with NRRL on Nov. 18, 2016. Methylobacterium sp. NRRL B-67741 was deposited with NRRL on Dec. 20, 2018. Methylobacterium sp. NRRL B-67742 was deposited with NRRL on Dec. 20, 2018. Methylobacterium sp. NRRL B-67743 was deposited with NRRL on Dec. 20, 2018. Methylobacterium sp. NRRL-B-67892 was deposited with NRRL on Nov. 26, 2019. Methylobacterium sp. NRRL-B-68032, NRRL-B-68033, and NRRL-B-68034 were deposited with NRRL on May 20, 2021.

Subject to 37 CFR §1.808(b), all restrictions imposed by the depositor on the availability to the public of the deposited material will be irrevocably removed upon the granting of any patent from this patent application.

### EXAMPLES

The following examples are given for purely illustrative and non-limiting purposes of the present invention.

### Example 1. Effects of Methylobacterium Strain ISO110 (NRRL B-50938) Treatment of Spinach on Mineral Nutrient Content of Harvested Leaves

Spinach seeds were treated with Methylobacterium strain ISO110 at a rate of 10⁶ CFU per seed and grown in soil mix (Fick’s garden mix soil) in 15 flats (26 seeds per flat) in a greenhouse in parallel with 15 flats of untreated spinach seeds. Flats were thinned to contain no less than 20 plants. At 28 days after planting (approximately 7 true leaves), 15 or more plants per flat were chosen randomly and shoots were collected by cutting one inch above the soil line. The shoots were incubated in sample bags at 45° C. for 4 days to dry and analyzed for macronutrient and micronutrient content. A single-tailed unequal variances (Welch’s) t-test was used to analyze the data to determine whether treatment with ISO110 resulted in a significant increase in nutrient content. Methylobacterium ISO110 significantly enhanced foliar content of three nutrients: nitrogen (N), magnesium (Mg), and iron (Fe). Other nutrients elevated over the UTC by treatment with ISO110 were copper, calcium, potassium and sulfur. Levels of zinc, boron, phosphorus and manganese were lower in ISO110 treated plants in comparison to control untreated plants.

Percent differences between the ISO110 treatment and the UTC treatment for macro- and micronutrients measured in this experiment are shown in Table 2. P-values were estimated using Student’s t-test. Results showing a difference at p < 0.1 are noted in italics.

TABLE 2

| Nutrient type | Nutrient (units) | TS401 value | UTC value | % difference from UTC | Contrastp-value v. UTC |
|---|---|---|---|---|---|
| Macronutrient | Nitrogen (%) | 5.454 | 4.855 | +12.3% | 0.023 |
| | Phosphorus (%) | 0.506 | 0.556 | -8.9% | 0.20 |
| | Potassium (%) | 12.2 | 12.0 | +2.0% | 0.48 |
| | Calcium (%) | 0.92 | 0.88 | +4.6% | 0.41 |
| | Magnesium (%) | 1.27 | 1.09 | +16.2% | 0.045 |
| | Sulfur (%) | 0.463 | 0.456 | +1.5% | 0.59 |
| Micronutrient | Zinc (ppm) | 129.1 | 151.1 | -14.6% | 0.060 |
| | Manganese (ppm) | 56 | 57 | -1.8% | 0.69 |
| | Iron (ppm) | 110.1 | 96.9 | +13.006% | 0.086 |
| | Copper (ppm) | 10.9 | 10.2 | +7.0% | 0.18 |
| | Boron (ppm) | 53.7 | 59.4 | -9.7% | 0.033 |

### Example 2. Assay for Methylobacterium Effect on Micronutrient Content and Increased Early Growth in Hydroponic System

The experiment is conducted using a randomized complete block design. An experiment with 3 treatment levels to compare the biomass of plants following seed treatment with 2 Methylobacterium strains and water to a control treated with only water is conducted as follows for testing growth enhancement effects of Methylobacterium isolates. The experiment has an n=10, and is laid out in 10 completely randomized blocks. Each experimental unit consists of 24 individual plants grown on a quarter (3×8 cubes) sheet of horticube and bulked for biomass. Ten horticube sheets (104 cell Oasis HorticubeXL™, single dibble; Smithers-Oasis North America, Kent, OH, USA) are each divided into four 3×8 cube pieces, and 30 pieces are placed into their own clean 1020 mesh tray. The horticube pieces are completely saturated with UV filtered R.O. water, and one seed (lettuce or spinach) is placed in each dibble (pre-formed seed hole) of the horticubes. Seeds are inoculated by applying 10⁶ CFU of a Methylobacterium strain to be tested directly to each seed.

Seeds are allowed to grow undisturbed at 23-25° C. and 14 hour days. Plants are broadcast watered and fertilized (15-16-17) on Mondays, Wednesdays and Fridays. Plants are watered with UV filtered RO water on all other days. Fourteen days after planting (approximately 2 true leaf stage), the shoot portion of each plant is harvested by cutting directly below the cotyledon and all the shoots from the same tray are bulked together. The shoots are allowed to dry in an oven at 45° C. for at least 3 days and the bulked shoots from each sheet/tray weighed to identify Methylobacterium strains that increase shoot biomass in lettuce or spinach following seed treatment. Shoots may be from the same samples as measured to determine biomass or from a separate experiment conducted as described above.

Results of analysis of the effect of treatment with various Methylobacterium strains on enhanced early growth of 2 true leaf stage lettuce and spinach plants as described above are provided in Tables 3 and 4 below. Lettuce results in Table 3 are from biomass data only. Data are combined results from at least 3 independent repetitions of an experiment with a given isolate. Contrast p-values are taken from Student’s t-test post hoc to a linear mixed model. The lettuce results in Table 3 show that using LGP2002, LGP2001, LGP2010, LGP2012, LGP2000, LGP2009, LGP2006, LGP2011, LGP2007, LGP2004, LGP2025, LGP2026, LGP2021, LGP2020, LGP2017, LGP2028, LGP2029, LGP2030, LGP2019, LGP2031, LGP2016, LGP2033, LGP2034, LGP2022, LGP2023, and a combination of LGP2002 and LGP2015 results in a positive percent growth enhancement over control.

TABLE 3

Lettuce Growth Measurement

| Treatment | Percent growth enhancement over Control | Contrast p-value vs. Control |
|---|---|---|
| LGP2002 | +2.9% | 0.24 |
| LGP2001 | +8.4% | 0.035 |
| LGP2010 | +9.7% | 0.0038 |
| LGP2012 | +4.3% | 0.0025 |
| LGP2000 | +7.0% | 0.035 |
| LGP2009 | +9.6% | 0.017 |
| LGP2006 | +5.3% | 0.44 |
| LGP2011 | +2.7% | 0.24 |
| LGP2007 | +9.5% | 0.0043 |
| LGP2004 | +1.4% | 0.56 |
| LGP2024 | -10.5% | 0.14 |
| LGP2025 | +4.1% | 0.53 |
| LGP2026 | +8.2% | 0.23 |
| LGP2021 | +7.8% | 0.0007 |
| LGP2027 | -3.0% | 0.66 |
| LGP2020 | +1.8% | 0.26 |
| LGP2017 | +1.2% | 0.14 |
| LGP2028 | +1.3% | 0.24 |
| LGP2029 | +5.3% | 0.0038 |
| LGP2030 | +2.8% | 0.06 |
| LGP2019 | +2.7% | 0.22 |
| LGP2031 | +0.3% | 0.64 |
| LGP2032 | -7.6% | 0.27 |
| LGP2016 | +1.7% | 0.89 |
| LGP2033 | +2.0% | 0.13 |
| LGP2034 | +4.8% | 0.011 |
| LGP2022 | +10.9% | 0.011 |
| LGP2023 | +4.6% | 0.047 |
| LGP2002 + LGP2015 | +5.3% | 0.0043 |

Spinach results in Table 4 are based on image data as a proxy for aboveground biomass. Data are combined results from 2 independent repetitions of experiment. Contrast p-values are taken from Student’s t-test post hoc to a linear mixed model. The spinach results in Table 4 show that using LGP2001, LGP2010, LGP2009, LGP2021, LGP2022, LGP2023, and a combination of LGP2002 and LGP2015 results in a positive percent growth enhancement over control.

TABLE 4

Spinach Growth Measurement

| Treatment | Percent growth enhancement over Control | Contrastp-value vs. Control |
|---|---|---|
| LGP2001 | +2.7% | 0.33 |
| LGP2010 | +2.0% | 0.48 |
| LGP2009 | +0.7% | 0.81 |
| LGP2021 | +0.8% | 0.78 |
| LGP2022 | +4.0% | 0.15 |
| LGP2023 | +1.9% | 0.49 |
| LGP2002 + LGP2015 | +1.4% | 0.62 |

### Example 3. Detection or Identification of Methylobacterium Strains, Variants and Derivatives

Assays are disclosed for detection or identification of specific Methylobacterium strains and closely related derivatives. Genomic DNA fragments unique to a Methylobacterium strain are identified and qPCR Locked Nucleic Acid (LNA) based assays are developed.

Genomic DNA sequences of Methylobacterium strains are compared by BLAST analysis of approximately 300bp fragments using a sliding window of from 1-25 nucleotides to whole genome sequences of over 1000 public and proprietary Methylobacterium isolates. Genomic DNA fragments are identified that have weak BLAST alignments, indicative of approximately 60-95% identity over the entire fragment, to corresponding fragments of a Methylobacterium of interest. Fragments from the LGP2015 genome corresponding to the identified weak alignment regions were selected for assay development and are provided as SEQ ID NOS: 1-3.

TABLE 5

Unique Fragment Sequences of LGP2015

| Fragment | SEQ ID NO | Sequence |
|---|---|---|
| ref1_135566 | 1 | ACGGTCACCCCACGGACTGGGCGAGTACCTCACCGGTGTTCTA TCATAACGCCGAGTTAGTTTTCGACCGTCCCTTATGCGATGTA CCACCGGTGTCGGCAGCCGATTTCGTCCCACCGGGAGCTGGCG TTCCGGTTCAGACCACCATCATCGGTCACGATGTCTGGATTGG ACACGGGGCCTTCATCTCCCCCGGCGTGACTATAGGAAACGGC GCGATCGTCGGGGCCCAGGCGGTCGTCACAAGAGATGTCCCA CCCTATGCGGTAGTTGCTGGCGTCCCCGCGACCGTACGACGAT |
| ref1_135772 | 2 | CCAATAAAAGCGTTGGCCGCCTGGGCAACCCGATCCGAGCCT AAGACTCAAAGCGCAAGCGAACACTTGGTAGAGACAGCCCGC CGACTACGGCGTTCCAGCACTCTCCGGCTTTGATCGGATAGGC ATTGGTCAAGGTGCCGGTGGTGATGACCTCGCCCGCCGCAAGC GGCGAATTACTCGGATCAGCGGCCAGCACCTCGACCAAGTGT CGGAGCGCGACCAAAGGGCCACGTTCGAGGACGTTTGAGGCG CGACCAGTCTCGATAGTCTCATCGTCGCGGCGAAGCTGCACCT CGA |
| ref1_169470 | 3 | CGATGGCACCGACCTGCCATGCCTCTGCCGTCCGCGCCAGAAT GGTAAAGAGGACGAAGGGGGTAAGGATCGTCGCTGCAGTGTT GAGCAGCGACCAGAGAAGGGGGCCGAACATCGGCATCAAACC TCGATTGCCACTCGGACGCGAAGCGCGTCTTGAAGGAGGGAT GGAAGCGAAACGGCCGCAGAGTAACCGCCGACGAAAGATTGC ACCCCTCATCGAGCAGGATCGGAGGTGAAGGCAAGCGTGGGT TATTGGTAAGTGCAAAAAATATAATGGTAGCGTCAGATCTAGC GTTC |

Regions in SEQ ID NOS: 1-3 where corresponding regions in other Methylobacterium strains were identified as having one or more nucleotide mismatches from the LGP2015 sequence were selected, and qPCR primers designed using Primer3 software (Untergasser et al. (2012), Koressaar et al. (2007)) to flank the mismatch regions, have a melting temperature (Tm) in the range of 55-60 degrees, and to generate a PCR DNA fragment of approximately 100 bp. The probe sequence was designed with a 5′ FAM reporter dye, a 3′ Iowa Black FQ quencher, and contains one to six LNA bases (Integrated DNA Technologies, Coralville, Iowa). At least 1 of the LNA bases is in the position of a mismatch, while the other LNA bases are used to raise the Tm. The Tm of the probe sequence is targeted to be 10 degrees above the Tm of the primers.

Primer and probe sequences for detection of specific detection of LGP2015 are provided as SEQ ID NOS: 4-12 in Table 6. Each of the probes contains a 5′ FAM reporter dye and a 3′ Iowa Black FQ quencher.

TABLE 6

Primer and Probe Sequences for Specific Detection of LGP2015

| Primer/Probe | SEQ ID NO | Sequence^{∗} |
|---|---|---|
| LGP2015_ref1_135566_forward | 4 | CCTCACCGGTGTTCTATCATAAC |
| LGP2015_ref1_135566_reverse | 5 | CCGATGATGGTGGTCTGAAC |
| LGP2015_ref1_135566_probe | 6 | CGTCCCTTATGCGATGTACCA |
| LGP2015_ref1_135772_forward | 7 | GATCCGAGCCTAAGACTCAAAG |
| LGP2015_ref1_135772_reverse | 8 | GACCAATGCCTATCCGATCAA |
| LGP2015_ref1_135772_probe | 9 | AACACTTGGTAGAGACAGCC |
| LGP2015_ref1_169470_forward | 10 | AAGGAGGGATGGAAGCGAAAC |
| LGP2015_ref1_169470_reverse | 11 | ATAACCCACGCTTGCCTTC |
| LGP2015_ref1_169470_probe | 12 | CGCAGAGTAACCGCCGACGAA |

^{∗}Bold and underlined letters represent the position of an LNA base

### Use of Primer/Probe Sets on Isolated DNA to Detect LGP2015 and Distinguish From Related Methylobacterium Isolates

Each 10 µl qPCR reaction contains 5 µl of Quantabio PerfeCTa qPCR ToughMix 2x Mastermix, Low ROX from VWR, 0.5 µl of 10 µM forward primer, 0.5 µl of 10 µM reverse primer, 1 µl of 2.5 µM probe, 1µl nuclease free water and 2 µl of DNA template. Approximately 1 ng of DNA template is used per reaction. The reaction is conducted in a ThermoFisher QuantStudio™ 6 Flex Real-Time PCR System with the following program: 95° C. for 3 min, then 40 cycles of 95° C. for 15 sec and 60° C. for 1 min. The analysis software on the PCR instrument calculates a threshold and Ct value for each sample. Each sample was run in triplicate on the same qPCR plate. A positive result is indicated where the delta Ct between positive and negative controls is at least 5.

Use of the three primer/probe sets to distinguish LGP2015 from closely related isolates by analysis of isolated DNA is shown in Table 7 below. The similarity score shown for the related isolates takes into account both the average nucleotide identity and the alignment fraction between the isolates and LGP2015. One of the tested strains, LGP2035, was used as an additional positive control. LGP2035 is a clonal isolate of LGP2015 which was obtained from a culture of LGP2015, which was confirmed by full genome sequencing as identical to LGP2015, and which scored positive in all three reactions. The similarity score of greater than 1.000 for this strain is likely the result of a slightly different assembly of the genome for this isolate compared to LGP2015. The delta Ct of approximately 15 or more between the LGP2015 and LGP2035 isolates and the water only control is consistent with the sequence confirmation of the identity of these isolates. Analysis of other isolates that are less closely related to LGP2015 results in delta Ct values similar to those for the water only control.

TABLE 7.

| LGP# | Similarity score to LGP2015 | Average Ct Value | | |
|---|---|---|---|---|
| | | Ref1_135566 | Ref1_135772 | Ref1_169470 |
| LGP2035 | 1.005 | 21.08 | 21.31 | 20.35 |
| LGP2015 | 1 | 21.97 | 22.62 | 22.08 |
| LGP2036 | 0.181 | No Ct | 37.85 | >37.91 |
| LGP2037 | 0.87 | >36.8 | >38.31 | No Ct |
| LGP2038 | 0.88 | >38.36 | >38.36 | >38.44 |
| LGP2039 | 0.894 | No Ct | >37.47 | >38.13 |
| LGP2031 | 0.852 | 37.81 | No Ct | 37.97 |
| LGP2040 | 0.862 | 37.94 | 38.37 | >38.35 |
| LGP2034 | 0.807 | 38.44 | No Ct | No Ct |
| LGP2041 | 0.894 | 38.77 | No Ct | >37.91 |
| LGP2042 | 0.872 | 37.64 | 37.20 | 37.96 |
| H₂O only | | >38.14 | >35.92 | >37.12 |

### Use of Primer/Probes for Detection of LGP2015 on Treated Plant Materials

For detection of LGP2015 foliar spray treatment on corn: Untreated corn seeds were planted in field soil in the growth chamber and watered with non-fertilized R.O. water. After plants germinated and grew for approximately 3 weeks, they were transferred to the greenhouse. At V5 stage, plants were divided into 3 groups for treatment: foliar spray of LGP2015, mock foliar spray, and untreated. Plants receiving the foliar spray of LGP2015 were treated with 10x glycerol stock at the rate of 71.4 µl per plant using Solo sprayers. This converts to the rate of 10 L/acre in the field. Mock treated plants were sprayed with 71.4 µl water/plant. Untreated plants received no foliar spray treatment. Leaves were harvested two weeks after foliar spray treatment into sterile tubes and DNA from bacteria on the harvested leaves is isolated as described above. Each experiment was grown at least 2 times. As shown in Table 8, LGP2015 is detected on leaves harvested from corn plants treated by a foliar spray application of the Methylobacterium strains using all 3 primer probe sets, as demonstrated by delta Ct values of approximately 10 between the sample and the negative controls.

TABLE 8

Average Ct Value

| Treatment | Ref1_135566 | Ref1_135772 | Ref1_169470 |
|---|---|---|---|
| Control (no application) | 32.43 | 32.10 | 31.55 |
| Control (mock application) | 35.54 | 35.34 | 34.80 |
| LGP2015 (10L/acre equivalent) | 23.36 | 22.88 | 22.66 |

The above results demonstrate the use of genome specific primers and probes to detect Methylobacterium strain LGP2015 on various plant tissues following treatment with the strains and provide methods to distinguish LGP2015 from closely related isolates. Similar methods are developed for additional Methylobacterium strains, LGP2002, LGP2019, LGP2018 and LGP2017 using target sequence fragments and primer/probe pairs as shown in the Tables below.

TABLE 9

Target Fragment Sequences of LGP2002

| Fragment | SEQ ID NO | Sequence |
|---|---|---|
| ref4_930 | 13 | GCAAAACGACCTAATAGTTCTACAGCGGCATGCGCCAAGT CAGCGCGGTGAACAGTATACCTGGGAGCAACTTGTCCTCC GAAACCCACATAAAACAAATTACTCCTGGCAGTGCCCAGT CCATCAAAATCGAATACAATATTTCTCGAGGAGGCATCTGT AATAGCCTGCCAAAGCAACAAAGCTATGGCGCCGTTATGA CTTTCATTGCTTCTGGTAGACATAAAATAATATGCCGATTT GTGATCCCAAATGTAGAATATTGCCGCATCAATTGCGCCAA GTTTATTTCGGATCGAT |
| ref1_142021 | 14 | GGCGCCAACGGTATGATCGCATGATTTTCCTGCGGCATAGC TTGCGGGAATGGCGTATTTGGCGCTCTCCTCAGGAATTTCT AAGGGCATACGCAGGAACTCTACAGCACTTTTACTGGTATT TTGTAGTGACAGCGGAGGAGGCTGGTGCTCAAGGTAATCG TGATGAAGTGATCCGGGCCATTCGGGGCGCGTTTCTAGTCT TTCCAATCCGCGCCCTGTACCACGTATTACGCCGGACCGGT CTGCGCCGCGCCGCCCTCTTGACCGCCCTAAATGTCTAAGA GCGTCTAACAAAGC |
| ref1_142636 | 15 | GACGATATCGCTCATCTTCACTGCATTGAAGCTGGTGCCGT ACTGCATAGGGATGAAAAAGTGATGCGGATAGACGGCTGA CGGGAAAGCGCCTGGTCGATCGAAGACTTTGCTGACGAGG TTGTGGTAGCCCCGGATATAGGCATCGAAGGCCGGGACGT TGATCCCATCCTTTGCCTTATCTTGACTGGCGTCGTCGCGTG CCGTCAGAACGGGCACGTCGCAGGTCATCGAGGCCAGCAC CTTGCGGAACACCTGCGTTCCGCCGTTGGGATTATCGACGG CGAACGCGGTGGCCGC |

TABLE 10

Primer and Probe Sequences for Specific Detection of LGP2002

| Primer/Probe | SEQ ID NO | Sequence^{∗} |
|---|---|---|
| LGP2002_ref4_930_forward | 16 | GTCCTCCGAAACCCACATAAA |
| LGP2002_ref4_930_reverse | 17 | CTACCAGAAGCAATGAAAGTCAT |
| LGP2002_ref4_930_probe | 18 | TCTGTAATAGCCTGCCAAAGCA |
| LGP2002_ref1_142021_forward | 19 | GGCTGGTGCTCAAGGTAAT |
| LGP2002_ref1_142021_reverse | 20 | ACATTTAGGGCGGTCAAGAG |
| LGP2002_refl_142021_probe | 21 | ATGAAGIGATCCGGGCCAT |
| LGP2002_ref1_142636_forward | 22 | CCGTACTGCATAGGGATGAAA |
| LGP2002_ref1_142636_reverse | 23 | TAAGGCAAAGGATGGGATCAA |
| LGP2002_refl_142636_probe | 24 | TTGCTGACGAGGTTGTGGTAG |

^{∗}Bold and underlined letters represent the position of an LNA base

TABLE 11

Target Fragment Sequences of LGP2019

| Fragment | SEQ ID NO | Sequence |
|---|---|---|
| ref1_458355 | 25 | CAACTATGTAGACCCGACGGTGCGATTTCACTTCGCAAAGCCG CAGGGCAGCACCCTTGCGCTCAATGTTGACGCCAGCGTGATCT ATACTATTACCGTCACGCACACGCAGGGCGGCGTACAGATTCA TCGCGAGAGTAAGAACCACCATCAGACCATCACGCGCAGCGA CCTGAGCAAGCAGTTCGGCGTTGGTGTGGCCGACCAGCTGAC GCGCGATCAGGTCATGAAGGTGATCGAGTCGGCATTTCGCGA CGCTACCCGCTAAGATCGGCGCCCACGAAACGCTACGAGACT AGG |
| ref1_459688 | 26 | AGCCGGCATCTTGTTCAAGGCGCTCACCTCGACGCCGACGCTG TAGGCGACTTGAGAGGGCGTCTCATATGAACGAAGCATCTTCG CGTAGAGAACCTTCTTGTTCTCCTGCGTGATGTTCGCTTTGCAG ACGTTGACTGCCGCCATGAACGCCGAAGCCTTGCGCGCTTCAT CGTAATCGCCTGCGAAGGCGGGTAGTGAAAAGCTTAGTGCAA TGGCAAACACAGCCGCCGAACGTCGCATGGTATCCGTCCCCG ATTGACGGCAGTGCCGCCATATCTCGGCTTTAGCAGAGCTGAT |
| ref1_3158527 | 27 | AACCTGCGCCGGCCGAGGTTTCGCGAGCCGTCGCCACGGGCA ACGCCTCGCCCGCGATGTGCAAAAAAGTCCCCGGCACTTCGCG CCGTCGTCCGATCCACGACCGCGAATTTCTCAACGAGTACAAG GTGCTTATGGGAGATCCGAGCGTCCGTCCCGGAGCCCGAGAC CGCGCGGCCCGAGTAATAGGCGAAAAAGACTCCTACTCCTCG GGCTTCTCGGGCCCCCTCAGCAACATCTACGCTTGCCGCCCAT CACCCTGGCGGGAGATCAGCGACGAGACACAGGCCCACTTCG CCC |

TABLE 12

Primer and Probe Sequences for Specific Detection of LGP2019

| Primer/Probe | SEQ ID NO | Sequence^{∗} |
|---|---|---|
| LGP2019_ref1_458355_forward | 28 | TTGACGCCAGCGTGATCTATAC |
| LGP2019_ref1_458355_reverse | 29 | GTGATGGTCTGATGGTGGTTCT |
| LGP2019_ref1_458355_probe | 30 | TATTACCGTCACGCACACG |
| LGP2019_ref1_459688_forward | 31 | CTTCGCGTAGAGAACCTTCTTGTT |
| LGP2019_ref1_459688_reverse | 32 | CTTCGCAGGCGATTACGATGAA |
| LGP2019_ref1_459688_probe | 33 | CGTGATGTTCGCTTTGCAGA |
| LGP2019_ref1_3158527_forward | 34 | CCGCGAATTTCTCAACGAGTACA |
| LGP2019_ref1_3158527_reverse | 35 | GCCCGAGGAGTAGGAGTCTTT |
| LGP2019_ref1_3158527_probe | 36 | AGGTGCTTATGGGAGATCCG |

^{∗}Bold and underlined letters represent the position of an LNA base

Use of the primer/probe sets to distinguish LGP2019 from closely related isolates by analysis of isolated DNA is shown in Table 13 below. The similarity score shown for the related isolates takes into account both the average nucleotide identity and the alignment fraction between the isolates and LGP2019. Two of the tested strains, LGP2043 and LGP2014, were used as additional positive controls since a similarity score of 1.00 indicates they are nearly identical to LGP2019. Consistently low Ct values from qPCR using LGP2019 as the DNA template and no detection in the water only control is consistent with the sequence confirmation of the identity of these isolates. Analysis of other isolates that are less closely related to LGP2019 results in no detection similar to those for the water only control.

TABLE 13

| LGP# | Similarity to LGP2019 | Average Ct Value | | |
|---|---|---|---|---|
| | | ref1_459688 | ref1_3158527 | ref1_458355 |
| LGP2019 | 1.00 | 22.39 | 24.09 | 23.10 |
| LGP2043 | 1.00 | 22.49 | 24.04 | 22.96 |
| LGP2014 | 1.00 | 22.49 | 23.86 | 22.90 |
| Strain A | 0.95 | UDT | UDT | UDT |
| Strain B | 0.94 | UDT | UDT | UDT |
| Strain C | 0.93 | UDT | UDT | UDT |
| Strain D | 0.93 | UDT | UDT | UDT |
| water only (neg control) | - | UDT | UDT | UDT |

TABLE 14

Target Fragment Sequences of LGP2017

| Fragment | SEQ ID NO | Sequence |
|---|---|---|
| ref1_1185955 | 37 | AGTCATTGATCAAGCAACCCCTATTGAGTTGGATATCGAAGGA TCAAGGTCGCGTCAATAGATGCATCTATCAGGCCAAATGTCGC TTTTCAAGAATGGCTCTTTCGAAGCTATCTTTATAATCGCTCGC CATTCTCTCATTACCAAAATCGACCTTAACTAGCTCGACATTG ATGCGAGCAGCTCCGGCAAACGAGGAGAGATTGACCTTAAAG GAATTGAACGCCTCAAGCAATTCAGACACATTACCAGGAGTG CTATAGCAACAACCAGACCCATATCGGTCAATAACCTCTTTTA |
| ref1_3282585 | 38 | CGCAAAACGATTTATCACTGCCATCTTGTTGTTTGATAACCCTT TTTTACCAGACGTTATGCTGGGCGAGAAAGAGGACTAGCAGA TCGGAGCGGTATCGCGATTTTTCGGTAGTTCGCGCCTACAACA GGATAAGATCCGATAGTGAAGCAACATGGCTGTTTTTTGATTT GTAAGTCAGCAACTTAAGCAGCCAGCCTATCTGCCGTCGCAGA CGCTTGAGGCATCGGGCAGCATCTTAGAAAAGGTGGCAGTAA TTGCCACAGCGGAACGTAGCGGCACGGATAAGCACGCAGGGT C |
| ref1_4194637 | 39 | CCCATCTGGACCCAATATCCCCTTCATCGACAATTCCCGAGTA AGTGTGGGTTCGAGGATTTCGCGAAACAGCCTTGTTCGTTCCT CCGGCCTTAAAATTGGCGTGCCGTCGGGAGATCGATAGGCATC CCTTACCTGCCTTTCGACCGCCGGCACACGCGCGCCGGTCGTC GTGTTCACGGCCACGGAATGGACGAAGGTGCGCCGCTCATTTC GCTCGTTTGCCGTCTCCACCATCCAGGAGGCCAGCAGGACGGT TTCGTCTCGACCGCCGGTCACACACACCGCAAGGGACTCAGG |

TABLE 15

Primer and Probe Sequences for Specific Detection of LGP2017

| Primer/Probe | SEQ ID NO | Sequence^{∗} |
|---|---|---|
| LGP2017_ref1_1185955_forward | 40 | TCGCTCGCCATTCTCTCATTAC |
| LGP2017_ref1_1185955_reverse | 41 | AGGTCAATCTCTCCTCGTTTGC |
| LGP2017_ref1_185955_probe | 42 | TCGACATTGATGCGAGCA |
| LGP2017_ref1_3282585_forward | 43 | TTCGCGCCTACAACAGGATAAG |
| LGP2017_ref1_3282585_reverse | 44 | CAGATAGGCTGGCTGCTTAAGTT |
| LGP2017_ref1_3282585_probe | 45 | TCCGATAGTGAAGCAACA |
| LGP2017_ref1_4194637_forward | 46 | GAGTAAGTGTGGGTTCGAGGATTT |
| LGP2017_ref1_4194637_reverse | 47 | AGGTAAGGGATGCCTATCGATCT |
| LGP2017_ref1_4194637_probe | 48 | CGGAGGAACGAACAAGGC |

^{∗}Bold and underlined letters represent the position of an LNA base

TABLE 16

Target Fragment Sequences of LGP2018

| Fragment | SEQ ID NO | Sequence |
|---|---|---|
| LGP2018_ref1_4871392 | 49 | ACCTGCTAAAATCACGTCCTCTCAGATTGAAAAAT CATTGAAGAAACGTGTCGAACGATTGCCGGGGATT ATGACGTTAGATCAATTGAAAAATACAAGCTTTGA AATTGAGTTACAGCCAAAAGATGCCCCGGATCCGG ACCCATCAGACTTCGGTGGCTAGTTCGAGCCAAAC TCGAACGTCGCCATGGCGCGCAAGTCGCAATACCA TTTCACAGCGCAGCGGTTATTTCGTTGTACACTGTA GCAATGCGTCGGCTTGCGCGCTTCCGCTGGCGATC AAAGGTCCGCCGATTTACG |
| LGP2018_ref1_1266930 | 50 | TCCCGAACATACAATGGAGGAAGCGTGTGGTAGGC CAATTTGTAACGAAATATGGCATCGGTCACGGCTC TCTCAATAAATTCGATCTCAAGTCTTCTGAACGAG CATGCCTCATCCTTATCCTGAGCGAACGCCTGCCA GTTTGCAGTCATTCCAACATACATAGCCAAAAAGG CGAGGTAGACCTTCATACGGGCACCTCAATCGTCC CCATTCGTTCAAGCTCCTTCAAGATAACAGCCGCA CCACATTGCTGAGATCGAAGATTCGGATCAAATAT TCCATCAAATTTATACTTTC |
| LGP2018_ref1_17614 | 51 | GCATCCTTTGCGCTCGCAGGCCTAAGGTCAAGCCC GGTTACTTCGTTTGGTAGAACGAGGTAGACGATGC CTAGTCTTAAGGTGGCCCATGTTAACCAACAGGGC CAGAACATGATTATAGTTCCGTTAGATGCCAACTT CGGTTACAAAACCGATGGTGAGCAGTCCGACATCA TGTTCGAAATACAGGACGCGGCGCGGTCCGCCGGT CTTGCGGGTGCCGTAGTAGCGTTCTGGCAGTCAGG TGGACAAACCCGTTTCCGGGGCCCGGCTCCGTGGC ACCCATTCCTTCGCAGCCTC |

TABLE 17

Primer and Probe Sequences for Specific Detection of LGP2018

| Primer/Probe | SEQ ID NO | Sequence^{∗} |
|---|---|---|
| LGP2018_ref1_4871392_forward | 52 | GCGCAAGTCGCAATACCATTTC |
| LGP2018_ref1_4871392_reverse | 53 | CGTAAATCGGCGGACCTTTGA |
| LGP2018_ref1_4871392_probe | 54 | CGCAGCGGTTATITCGTTG |
| LGP2018_ref1_1266930_forward | 55 | ACGAGCATGCCTCATCCTTATC |
| LGP2018_ref1_1266930_reverse | 56 | CGATTGAGGTGCCCGTATGAA |
| LGP2018_ref1_1266930_probe | 57 | TGCCAGTTTGCAGTCATTCC |
| LGP2018_ref1_17614_forward | 58 | CCCGGTTACTTCGTTTGGTAGAA |
| LGP2018_ref1_17614_reverse | 59 | CGAAGTTGGCATCTAACGGAACTA |
| LGP2018_ref1_17614_probe | 60 | TGGCCCATGTTAACCAACAG |

^{∗}Bold and underlined letters represent the position of an LNA base

### Use of Primer/Probes for Detection of LGP2019 on Treated Plant Materials

Detection of LGP2019 from in-furrow treated corn roots:

At planting, corn seeds in soil were drenched with LGP2019 and control strains from frozen glycerol stock to simulate in-furrow treatment. To obtain a final concentration of 10⁷ CFU/seed, 100 µl of each strain at 10⁸ CFU/ml is inoculated onto each seed placed in the dibble holes in soil. A ⅒ dilution series is made for lower concentration targets. For control treatment, 100 µl Milli-Q water is applied to each corn seed placed in the dibble holes in soil. Pots containing treated seeds are placed in a growth chamber for approximately two weeks and watered with unfertilized RO water every 1-2 days to keep soil moist. After 2 weeks of growth, roots of about 9 plants per replicate sample were harvested into sterile tubes. Each treatment had at least 2 replicate samples in each experiment, and each experiment was conducted at least 3 times.

DNA from bacteria on the harvested corn roots is isolated as follows. Individual roots are submerged in 20 mL of phosphate-buffered saline (PBS) (137 mM NaCl, 10 mM Phosphate, 2.7 mM KCl, and a pH of 7.4) in 50 mL conical tubes. Tubes are vortexed for 10 minutes, and then sonicated for 10 minutes. Root tissue is removed, and the remaining supernatant from multiple roots of the same sample are combined and centrifuged at 7500xg for 10 minutes. This process is repeated until there is one tube for each sample. The moist soil pellet is vortexed until it evenly coats the tube wall. Tubes are placed into a laminar flow hood with caps removed and open ends of the tubes facing the air blowers. Once dry, samples are stored at room temperature. 250 mg dried soil is used as input for DNA extraction using Qiagen DNeasy PowerSoil HTP 96 kit (Cat#12955-4) using manufacturer protocols.

Primers and probes for LGP2019 disclosed in Table 12 above are used in qPCR reactions to detect the presence of LGP2019 specific fragments provided in Table 11. Each 10 µl qPCR reaction contains 5 µl of Quantabio PerfeCTa qPCR ToughMix 2x Mastermix, Low ROX from VWR, 0.5 µl of 10 µM forward primer, 0.5 µl of 10 µM reverse primer, 1 µl of 2.5 µM probe, 1 µl nuclease free water and 2 µl of DNA template. Approximately 1 ng of DNA template is used per reaction. The reaction is conducted in a ThermoFisher QuantStudio™ 6 Flex Real-Time PCR System with the following program: 95° C. for 3 min, then 40 cycles of 95° C. for 15 sec and 60° C. for 1 min. The analysis software on the PCR instrument calculates a threshold and Ct value for each sample. Each sample is run in triplicate on the same qPCR plate. A positive result is indicated where the delta Ct between positive and negative controls is at least 5.

### Use of Primer/Probes for Detection of Variants of Additional Table 1 Methylobacterium Isolates

Variants of Methylobacterium isolates listed in Table 1 are identified by the presence of DNA fragments as described above. Unique fragments for use in such methods are provided in Table 18.

TABLE 18

| Strain | Fragment | SEQ ID NO | Sequence |
|---|---|---|---|
| LGP2001 | ref3_25009 | 61 | GCCCTTCTGTCAGGCGATATTGTATAATGGCGTT GCCCCAATAGAAGCAGCCATTCGTGCGAGGGCA GCAGCGACGCTAGGTCGAAAGAGCATCCTAATCT CGATCAAGATGCGACTGAGATTTCTGATGAAAAT ATCTAGACACAAGCAAAGCTGGTGAAATTACAA CGATCATGGCGACAATTGCGGCCAATTCGGCCGG AACTTGAAGGAACATAAAAATGAATATTACAAA TATACCGCAAAGCATGTAGAGTTGCTACACCAAG GGTCGGGACGTCCAAAAAAACTCACTGAGGA |
| LGP2001 | ref3_25219 | 62 | GGAACATAAAAATGAATATTACAAATATACCGC AAAGCATGTAGAGTTGCTACACCAAGGGTCGGG ACGTCCAAAAAAACTCACTGAGGAAGTCGACTG GAAGCACGAGGCGCCCCCCCCAGGAGCGGGGCG ACCGGCAAGGGGGCCCGCAATTGTCGCCATGATC GACCAGCTTAGGTAGGATCCTCTTTCGACCTAAC GAATGGCTGCTTCTATTGGGGCAACGCCATTATA CAATATCGCCTGACCATCTGGAACGCGGCCCGGT CCACCGGCAGGTTGGCGACGACAGCGTCGGAG |
| LGP2001 | ref1_4361220 | 63 | CGGCGTCGACCAGCCGGGCGAACTGCTTGGGCAT GCTCTCCCGCGACGCCGGCCACAGCCGCGTCCCC GTCCCTCCGCACAGGATCATCGGGTGGATTTGAA AGGCAAAACGGGACATCAGGATAGGCCGCTCAG GCGTTGGCGCTGAGGCGCTTGATGTCGGCGTCGA CCATCTCGGTGATCAGCGCCTCGAGGCTGGTCTC GGCCTCCCAGCCGAAGGTCGCCTTGGCCTTGGCG GGGTTGCCCAGCAGCACCTCGACCTCTGCCGGCC GGAACAGCGCCGGGTCGACGATCAGGTGG |
| LGP2001 | ref1_4602420 | 64 | CTGGACATGCGCCCACCCCGGCCAAGTCCGACCG CACCGGCAACCGCTCCTGTAGTCGTCGTCATCGT TCTCACCCCTGAGGCGGAGACCGTCCGCTAACGG GGTGTCTCAAGCAACCGTGGGGCGGAGGAACAC GCACGTAGTCGCGTTTCAAGGTTCGCACGAACGC CTCGGCCATGCCGTTGCTCTGCGGGCTCTCCAGC GGCGTCGTTTTTGGCACCAAACCAAGGTCGCGGG CGAAGCGGCGCGTGTCGCGGGGACTGTCAGGAA TTTCGTGTGGGGGCGGCCATAGTGGATCCG |
| LGP2004 | ref1_194299 | 65 | GGAAATCGGCTTCAAGTACGACGTCACGCCGGCC ATGCAGGTCACGGGTGCACTGTTCAATCTCGAGC GCGACAACCAGCCGTTCCCCTCGAACGTGGAGTC CGGCCTCGTCCTTGGCGCAGGTCAGACACGCACC CAGGGCGCGGAAATCGGCCTGGCCGGCTATCTAA CCGATTGGTGGCAGGTCTTTGGCGGCTACGCTTA TACCGAGGCACGCGTACTCTCGCCACTGGAAGAC GATGGAGACGTGATCGCAGCAGGTAATCTCGTCG GCAACGTTCCGCTAAATACTTTCAGTCT |
| LGP2004 | ref1_194305 | 66 | CGGCCTGGCCGGCTATCTAACCGATTGGTGGCAG GTCTTTGGCGGCTACGCTTATACCGAGGCACGCG TACTCTCGCCACTGGAAGACGATGGAGACGTGAT CGCAGCAGGTAATCTCGTCGGCAACGTTCCGCTA AATACTTTCAGTCTGTTCAACAAGTTCGATATCA ACGAGAATTTCTCCGTTGCTCTGGGCTATTACTAT CAGGATGCCAGCTTTGCCTCCTCAGACAATGCAG TGCGTTTGCCAAGTTATTCGCGGTTCGATGGCGG GTTGTTCTATCGATTCGACGAGTTGAC |
| LGP2004 | ref1_194310 | 67 | ACGTTCCGCTAAATACTTTCAGTCTGTTCAACAA GTTCGATATCAACGAGAATTTCTCCGTTGCTCTG GGCTATTACTATCAGGATGCCAGCTTTGCCTCCTC AGACAATGCAGTGCGTTTGCCAAGTTATTCGCGG TTCGATGGCGGGTTGTTCTATCGATTCGACGAGT TGACACGCGTTCAGCTTAGCGTCGAGAACATTTT CGACAGGCGTTACATCATCAACTCCAACAACAAC AACAACCTCACGCCTGGCGCGCCGAGAACAGTCC GCGTGCAATTGATCGCTCGGTTCTAAA |
| LGP2003 | ref1_86157 | 68 | AGCCCACAAGCCTGATGCACTTAACTACATCCTC TAATGTCGCGCCAATTTGCTTGGCGGCAGGGGAT GTTGTATCGTCATAGGCTTGTCTAACCGGAACTT GTTTGCCAATCTCTTTGGCGATCGCAACCGCCAT CTCGTGTTCGTCAACCATGTGCGCGTTCCTCTAAT TGCACTCATGGTGCCACGTGCACCTCCGATCGTC TCGTGTCTAGAATGAAGGTGGGAACAACCTTACA CAGGCTTTCGCGACGCGCGAATTTCTGGTTTCTCC GCCTCGGATGTGGGTTTGAGCGCTTC |
| LGP2003 | ref1_142469 | 69 | CTTTTCATTTGTCATGATCTCGACCAAGGTATTCA CGGCAAGCTCGGTCTGTTGCTTAGCAAGTGCCTG AACTTCGCGAACGATCGGCTCTCGACCCTTCGGG TTCGAGACCTGTCCCTTTTGAAAACCACGTGCCC TACACTTTTCGGGATCAAGGTGCGGGTTGGCTTT GGTCAAAATTCTCTGGCGTCCCATTACACGCCCT CCGCATCATCGTTCCCGCGAACGATCTGACCCCC GACTTCCGCGAGGAAGCGTGTGGCGTGATCCTCG AAGCGGAATGCCACCTCGAACTGTTCC |
| LGP2003 | ref1_142321 | 70 | CAGCAGCAAGCAGATCGTTGAAAACCGCTTGAA CCGCATCTTGATCGGGACCGGAACCAATCAGGTC ATCTAGGTAAACCGAGACGTAAACTCGTTTGCGC TCGGCATCTTTCAGAACGTCCGTGATGCCAGACC GCATTAGTACCATCGTCGCCAAGGCGGGCGACTG AACGAAGCCGATCGGCAGAGAGTAACGGGGACC GCCCCTAATCGGGTTGCGAACGCAAGACCACTTA GCAAAGGTTCGAGCACGGCCGAACTTCGCATGGT GGAGAGCCGCGGCAACACGGTTCCGTGATA |
| LGP2009 | ref1_153668 | 71 | TAGACATTCCAACAAACCGGCAAGAGGCTCGTCC TCACTCGAGGATTTGTTGGGACTTGCATGATGTC GAAGCGGAGCCGTTATGACCTGGGTGCGATCATG CGCCGAGCATGGGAGATGGCTCGGGAGGCGGCA TTCGCGGTTGGCGAGCGGGCACGGACTCACCTTG CTGCCGCGATGCGCAGCGCGTGGGCCGAAGCCA AGTTGGCACTCGCGCCCACGAAGACGGAGCAGG ATCGTCTCTCTCCGAGCGACATGATCGGACATGA GGACGCCTACCAAGGCCGGGTTCTAAAATAT |
| LGP2009 | ref1_3842117 | 72 | AAGATGGATACGACAAGCGCGATTACATTATTTG CGAAATAGATGGACAAATAAAAGACAAAGGACT GATGTATTTCCTTAAATCTGGACAAGTTGACCTCT TTCACATAGAAGTCACCACTCCCTTTGGGACAAT TTGGTGTCACGAAAACATAGAGGCCGAACTTCTT AGCTGAATTATCGCGCTCCGGGTTCTTATGCGGC TGAGTGAAGCGCGGGACAGCTTGCGAGCAGGGC CGCCAATGGCAGCCGGGATGACACAATGCTCGGT CTCCCGACGCTTCTTCAATCGGGAGCGCT |
| LGP2009 | ref1_3842278 | 73 | AGCTGAATTATCGCGCTCCGGGTTCTTATGCGGC TGAGTGAAGCGCGGGACAGCTTGCGAGCAGGGC CGCCAATGGCAGCCGGGATGACACAATGCTCGGT CTCCCGACGCTTCTTCAATCGGGAGCGCTTCGCA GCCCGGGGCGGCGCGCTCATGCGTCACGACCTGG GCCCTGCGCACCTTCGCGGCCCCGCCGTCCCGGC AGATCCCTGATGCCCCAAGTGGGCGGCCACTCCA TCAAAGAACCCCGGCCTGTGGCAGATCTCGTAGG CATACCGAGGTTCCGCAGTGCCCCCACC |
| LGP2020 | ref1_2810264 | 74 | ACCGAAGGCGTCCCCGGACACGAAGGCCTGAAA CACCATATCTGTGGCGATCAGGCCGACGTGGTCG CGGACTTCAACTGGCAGAGAATGCCAGGCCGCTT CGATTTCAGATGATACTGGTACGGACATAGGAGC GGCTTAGCTTTCTCAGTGCAAATGTGATTGATTCC GGCTCAAAAATGATCTTGATCGGACGAGACGTTT TCAATCCATGTCGTGTTGCCATCGCCGATCGGTG CGTCAAGAGACAGATGGCGCCGACCGTAGATAC GCGTTCGGGTTGCCCGCACCGCTTCTCCA |
| LGP2020 | ref1_322980 | 75 | GGAGGTGTGATCTGATGATGTGCTGGATGAAATT GGCGGTCGAGCACTTGTTCAGCTTGGCCAGCTCG ACGAGATCGGCGTGATGCTCGGCGTCGATCAGGA TGTTCAGCGAGACCGGACGTACGCAGGACTTGGT ATTAGCGCCGTTGCGCATCAGCTTGCAGCCTTGC TCTGCTTCTCAGCGTGCCGCGTCAGGATGACCCT GATGTAGCTGTTGAGGTTGATGCCGTAATAGCCT GCGGACTCTGTGAGATCCCGGCGAAGATCGTCGG CGAGGGTCAGGCGGATGGTGCTGGTCGG |
| LGP2020 | ref1_2785241 | 76 | AAGTAACCGCTCAACATGATCTTCAGCATGTTGT CCAACAGCAGGAGAATACATGTAATTCACCATGA CCGGCAAGCTGCGACTGGCCATTGCTTCCACCGC TTGAATGTAGCGATCGAATTTCGCAAAATCAGGG TGGAATGAAAATATCGAACCAAACTGCGAGCCTT GAATCCGTTCTGCAAAATTATCGAAAAATTTTCT TGGCCGACTGCCGTTCGAAAACATTCTTACGTTT ACATGCGGCCCGCCTGAAACAAGACAGTCTACCA GCTCTGGGAAATGGGGGTGAAGGGTCGG |

### Example 4. Analysis of Effects of Methylobacterium Strains on Nutrient Content of Plant Vegetative Tissues

Soybean seeds treated as described in Example 1 were grown in multiple field locations in the Midwestern United States in the summer of 2019 in parallel with untreated control soybean plants. Seeds from Canola and wheat were similarly treated and tested. For analysis of field grown corn plants, Methylobacterium strains were applied in-furrow at planting. Strains and strain combinations evaluated are shown in Table 19 below.

TABLE 19

| | |
|---|---|
| Crop | Methylobacterium strain(s) |
| Soybean (+ Rhizobia treatment) | LGP2009 |
| Soybean (+ Rhizobia treatment) | LGP2020 |
| Soybean (+ Rhizobia treatment) | LGP2016 |
| Soybean (+ Rhizobia treatment) | LGP2002+LGP2015 |
| Soybean | LGP2002 |
| Soybean | LGP2009 |
| Soybean | LGP2004 |
| Soybean | LGP2015 |
| Soybean | LGP2001 |
| Soybean | LGP2017 |
| Soybean | LGP2002+LGP2015 |
| Soybean | LGP2019 |

Preliminary analysis of soybean vegetative tissue indicates increased micronutrients were obtained by treatment with Methylobacterium strains, including increased boron in R1 stage vegetative tissue in soybean plants grown from ISO103 and ISO118-treated seeds, and increased iron in V6 stage vegetative tissue in soybean plants grown from ISO102-treated seeds.

ISO103, ISO118, ISO102, ISO117, ISO120, and ISO121 are tested to evaluate effects on micronutrient levels and growth enhancement of leafy green plants as described in Example 2, and on enhancement of growth and yield of row crops, such as corn, rice, soybean, canola and wheat.

### Example 5. Methylobacterium Growth Stimulation of Cannabis Plants

The ability of Methylobacterium isolates LGP2002, LGP2009 and LGP2019, to enhance rooting and growth of cannabis plants (Cannabis sativa L.) was evaluated as follows. Cuttings were taken from a mature plant and immersed for 2 hours in a suspension of Methylobacterium in water at a concentration of approximately 1 × 10⁶ CFU per ml. A control solution (water only) contained no Methylobacterium. The wounded stem portion of cuttings in both the control and Methylobacteirum treatments were then dipped in synthetic rooting hormone 0.3% indole-3-butyric acid (IBA) and inserted, stem down, into a potting media plug in a mult-plug tray. Fifty plants total, 10 of each of 5 different CBD oil cannabis varieties, were treated with each Methylobacterium isolate. After 2 weeks in the potting medium, plugs were non-destructively harvested and roots were scored using a visual rating scale of 1-5: 1 = between 0 and 20% visible roots; 2 = between 21 and 40% visible roots; 3 = between 41 and 60% visible roots; 4 = between 61 and 80% visible roots; 5 = between 81 and 100% visible roots.

Rooting scores for plants treated with the tested Methylobacterium isolates ranged from 3-3.4, compared to a score of 2.6 for the untreated control plants. Treatments with LGP2002 and LGP2019 resulted in increases that were significantly different from the control at p<0.05, and treatment with LGP2009 resulted in increases that were significantly different from the control at p<0.001.

The rooted plantlets were transplanted to the field. Aboveground biomass was harvested approximately thirteen weeks after transplanting, dried and the aboveground dry biomass determined. Treatment with three Methylobacterium isolates, LGP2002, LGP2009 and LGP2019, resulted in increased aboveground dry biomass in comparison to the untreated control plants. Treatment with LGP2009 resulted in an 18% increase in aboveground dry biomass, treatment with LGP2002 resulted in a 27% increase in aboveground dry biomass, and treatment with LGP2019 resulted in a 38% increase in aboveground dry biomass, a difference that was significantly different from the control at p<0.05. Enhanced rooting as the result of treatment with Methylobacterium isolates can lead to earlier transplanting of plantlets to the field without negatively impacting yield, thus resulting in decreased cycling time.

### Example 6. Methylobacterium Growth Stimulation of Cannabis Plants

The ability of Methylobacterium isolates LGP2000, LGP2001, LGP2002, LGP2003, LGP2004, LGP2005, LGP2006, LGP2007, LGP2008, LGP2009, LGP2010, LGP2011, LGP2012, LGP2013, LGP2014, LGP2015, LGP2016, LGP2017, LGP2018, LGP2019, LGP2020, LGP2021, LGP2022, and LGP2023 to enhance rooting and growth of cannabis plants (Cannabis sativa L.) are evaluated as follows. Cuttings are taken from a mature plant and immersed for 2 hours in a suspension of Methylobacterium in water at a concentration of approximately 1 × 10⁶ CFU per ml. A control solution (water only) contains no Methylobacterium. The wounded stem portion of cuttings in both the control and Methylobacteirum treatments are then dipped in synthetic rooting hormone 0.3% indole-3-butyric acid (IBA) and are inserted, stem down, into a potting media plug in a mult-plug tray. Fifty plants total, 10 of each of 5 different CBD oil cannabis varieties, are treated with each Methylobacterium isolate. After 2 weeks in the potting medium, plugs are non-destructively harvested and roots were scored using a visual rating scale of 1-5: 1 = between 0 and 20% visible roots; 2 = between 21 and 40% visible roots; 3 = between 41 and 60% visible roots; 4 = between 61 and 80% visible roots; 5 = between 81 and 100% visible roots.

Rooting scores for plants treated with the tested Methylobacterium isolates are determined as compared to the untreated control plants.

The rooted plantlets are transplanted to the field. Aboveground biomass is harvested approximately thirteen weeks after transplanting, dried and the aboveground dry biomass is determined.

### Example 7. Increases in Rice Yield by Application of Methylobacterium

Rice field trials were conducted at three locations, all near Humphrey, AR, for the purpose of evaluating the effects of three Methylobacterium isolates applied as a seed treatment. Treatments included each Methylobacterium isolate and an untreated control applied to rice seeds with and without a base treatment of insecticide only (active ingredient Clothiandin). The trial was conducted using a Randomized Complete Block Design (RCBD) with 4 reps per location. ISO117 (NRRL B-67341), ISO120 (NRRL B-67743), and ISO118 (NRRL B-67741) were applied to rice seeds at a target concentration of 10⁶ CFU/seed.

The Methylobacterium isolates increased yield in rice field trials as compared to the untreated control both with and without insecticide treatment.

TABLE 20

Mean yield (Bu/A) Increase over control and percent increase shown

| Treatment | UTC | ISO117 | | ISO120 | | ISO118 | |
|---|---|---|---|---|---|---|---|
| Without insecticide treatment | 143.8 | 150.1 | +6.3 (4.3%) | 156.2 | +12.4 (8.6%) | 152.4 | +8.6 (6.0%) |
| With insecticide treatment | 151.8 | 164.3 | +12.5 (8.2%) | 155.4 | +3.6 (2.4%) | 158.2 | +6.4 (4.2%) |

(Bold italics indicates a significant difference at p < 0.05 using Fisher’s LSD test.)

When introducing elements of the present invention or the preferred embodiments(s) thereof, the articles “a”, “an”, “the” and “said” are intended to mean that there are one or more of the elements. The terms “comprising”, “including” and “having” are intended to be inclusive and mean that there may be additional elements other than the listed elements.

In view of the above, it will be seen that the several objects of the invention are achieved and other advantageous results attained.

As various changes could be made in the above compositions, methods and processes without departing from the scope of the invention, it is intended that all matter contained in the above description and shown in the accompanying drawings shall be interpreted as illustrative and not in a limiting sense.

Having described the invention in detail, it will be apparent that modifications and variations are possible without departing from the scope of the invention defined in the appended claims.

### REFERENCES

Green, P.N. 2005. Methylobacterium. In Brenner, D.J., N.R. Krieg, and J.T. Staley (eds.). “Bergey’s Manual of Systematic Bacteriology. Volume two, The Proteobacteria. Part C, The alpha-, beta-, delta-, and epsilonproteobacteria.” Second edition. Springer, New York. Pages 567-571.

Green, P.N. 2006. Methylobacterium. In Dworkin, M., S. Falkow, E. Rosenberg, K.-H. Schleifer, and E. Stackebrandt (eds.). “The Prokaryotes. A Handbook on the Biology of Bacteria. Volume 5. Proteobacteria: Alpha and Beta Subclasses.” Third edition. Springer, New York. Pages 257-265.

Green, P.N. and Ardley, J.K. 2018. Review of the genus Methylobacterium and closely related organisms: a proposal that some Methylobacterium species be reclassified into a new genus, Methylorubrum gen. nov. Int J Syst Evol Microbiol. 2018 Sep;68(9):2727-2748. doi: 10.1099/ijsem.0.002856 .

Konstantinidis K. T., Ramette A., Tiedje J. M.. ( 2006;). The bacterial species definition in the genomic era.. Philos Trans R Soc Lond B Biol Sci 361:, 1929-1940.

Lidstrom, M.E. 2006. Aerobic methylotrophic prokaryotes. In Dworkin, M., S. Falkow, E. Rosenberg, K.-H. Schleifer, and E. Stackebrandt (eds.). “The Prokaryotes. A Handbook on the Biology of Bacteria. Volume 2. Ecophysiology and biochemistry.” Third edition. Springer, New York. Pages 618-634.

Sy, A., Giraud, E., Jourand, P., Garcia, N., Willems, A., De Lajudie,P., Prin, Y., Neyra, M., Gillis, M., Boivin-Masson,C., and Dreyfus, B. 2001. Methylotrophic Methylobacterium Bacteria Nodulate and Fix Nitrogen in Symbiosis with Legumes. Jour. Bacteriol. 183(1):214-220.

What is claimed is:

1. A method for enhancing early plant growth that comprises:
(a) applying a composition to a plant, plant part or seed, wherein the composition comprises Methylobacterium LGP2022, LGP2023, LGP2021, or a variant thereof; and,
(b) growing the plant to at least the two leaf stage, thereby enhancing early plant growth in comparison to an untreated control plant that had not received an application of the composition or in comparison to a control plant grown from an untreated seed that had not received an application of the composition.

2. The method of claim 1, wherein the composition is applied to a seed.

3. The method of claim 1 or 2, wherein said plant is a leafy green plant.

4. The method of claim 3, wherein said leafy green plant is selected from the group consisting of spinach, lettuce, beets, swiss chard, watercress, kale, collards, escarole, arugula, endive, bok choy, and turnips.

5. The method of claim 3 or 4, wherein said leafy green plant is cultivated for production of microgreens and/or herbs.

6. The method of any one of claims 1-5, wherein said plant is cultivated in a hydroponic or aeroponic growth system.

7. The method of any one of claims 1-6, wherein said composition further comprises at least one additional component selected from the group consisting of an additional active ingredient, an agriculturally acceptable adjuvant, and an agriculturally acceptable excipient.

8. A composition comprising a fermentation product comprising a Methylobacterium strain, wherein said fermentation product is essentially free of contaminating microorganisms, and wherein the Methylobacterium strain is selected from the group consisting of LGP2022, LGP2023, LGP2021, and variants thereof.

9. The composition of claim 8, wherein said composition further comprises at least one additional component selected from the group consisting of an additional active ingredient, an agriculturally acceptable adjuvant, and an agriculturally acceptable excipient.

10. A plant, plant part or seed at least partially coated with the composition of claim 8 or 9.

11. A method for enhancing plant growth and/or rooting of a cannabis plant that comprises:
(a) treating a cannabis plant, plant part or seed with a composition comprising one or more Methylobacterium isolates and
(b) growing the treated plant or growing a plant from the treated plant part or seed to allow production of a rooted plant, wherein cannabis plant growth and or rooting is increased in comparison to an untreated control plant that had not received an application of the composition or in comparison to a control plant grown from an untreated plant part or seed that had not received an application of the composition.

12. The method of claim 11, wherein said composition comprises a Methylobacterium isolate selected from the group consisting of LGP2002, LGP2009, LGP2019, and a variant of LGP2002, LGP2009 or LGP2019.

13. The method of claim 11 or 12, wherein said plant part is a cutting from a cannabis plant.

14. The method of claim 13, wherein said cutting is treated by immersion in a Methylobacterium suspension.

15. The method of claim 14, wherein said Methylobacterium is present in said suspension at a concentration of greater than 1 × 10³ CFU per milliliter.

16. The method of any one of claims 11-15, wherein said rooted plant is transplanted to a field, and wherein the cycling time for production of a mature cannabis plant is decreased in comparison to a control cannabis plant grown from an untreated cutting.

17. A cannabis plant, part or seed that is at least partially coated with a composition comprising a Methylobacterium isolate selected from the group consisting of LGP2002, LGP2009, LGP2019, and a variant of LGP2002, LGP2009 or LGP2019, wherein said cannabis plant or a cannabis plant grown from said cannabis plant part or seed demonstrates enhanced plant growth or rooting, or decreased cycling time from cutting to mature plant, in comparison to a control cannabis plant that was not treated with said Methylobacterium or a cannabis plant grown from a control cannabis plant part or seed that was not treated with said Methylobacterium.

18. A leafy green plant or plant part having increased levels of one or more mineral nutrients and/or vitamins, wherein said plant or plant part is harvested from a cultivated plant grown from a Methylobacterium-treated seed or seedling, and wherein said Methylobacterium provides for increased levels of one or more mineral nutrients and/or vitamins.

19. The leafy green plant or plant part of claim 18, wherein said cultivated plant is is selected from the group consisting of spinach, lettuce, beets, swiss chard, watercress, kale, collards, escarole, arugula, endive, Bok choy, and turnips a spinach plant or plant part.

20. The leafy green plant or plant part of claim 18 or 19, wherein said Methylobacterium is ISO110 or a variant thereof.

21. The leafy green plant or plant part of any one of claims 18-20, wherein said plant or plant part has increased levels of one or more mineral nutrients selected from the group consisting of nitrogen, magnesium, and iron.

22. The method of any one of claims 1-7 or 11-16, wherein said enhanced growth results in a plant trait improvement to said plant selected from increased biomass production, decreased cycle time, increased rate of leaf growth, increased rate of root growth, and increased seed yield.

23. A method for identifying a Methylobacterium isolate that increases the content of one or more mineral nutrients and/or vitamins in a leafy green plant, said method comprising:
(i) treating a leafy green plant seed or leafy green plant seedling with at least a first Methylobacterium isolate;
(ii) cultivating said seed or seedling to obtain a treated plant having at least two true leaves;
(iii) harvesting the plant, plant shoot, or one or more true leaves from said treated plant;
(iv) analyzing the harvested plant, shoot or plant leaves to determine the mineral nutrient and/or vitamin content; and
(v) selecting a Methylobacterium isolate that increases the content of at least one mineral nutrient or vitamin in the treated plant, plant shoot or plant leaves in comparison to a plant, plant shoot or plant leaves harvested from an untreated control plant, or from a plant, plant shoot or plant leaves harvested from a plant treated with a second Methylobacterium isolate.

24. The method of claim 23, wherein seeds and/or plants are separately treated with a two or more different Methylobacterium isolates in step (i), and separately cultivated, harvested and analyzed in steps (ii) through (iv) to determine the mineral nutrient and/or vitamin content in the plants, shoots or one or more plant leaves in comparison to the other Methylobacterium isolates and, optionally, to an untreated control plant.

25. The method of claim 23, wherein seeds and/or plants are separately treated with three or more distinct Methylobacterium strains or combinations of distinct Methylobacterium strains in step (i), and said treated seeds or seedlings are separately cultivated, harvested and analyzed in steps (ii) through (iv) to determine the mineral nutrient and/or vitamin content in the plant, shoot or one or more plant leaves in comparison to the other distinct Methylobacterium treatments and, optionally, to an untreated control plant.

26. The method of any one of claims 23-25, wherein one or more of said distinct Methylobacterium strains has enhanced colonization efficiency.

27. The method of any one of claims 23-26, wherein said leafy green plant is cultivated in a hydroponic system or an aeroponic system.

28. The method of any one of claims 23-27, wherein said Methylobacterium strain selected in (v) as increasing the content of at least one mineral nutrient or vitamin also imparts a trait improvement to said leafy green plant selected from increased biomass production, decreased cycle time, increased rate of leaf growth, decreased time to develop two true leaves, increased rate of root growth, and increased seed yield.

29. The method of any one of claims 23-28, wherein said leafy green plant is selected from the group consisting of spinach, lettuce, beets, swiss chard, watercress, kale, collards, escarole, arugula, endive, bok choy, and turnips.

30. The method of any one of claims 23-29, wherein said leafy green plant is cultivated for production of microgreens and/or herbs.

31. The method of any one of claims 23-30, wherein said leafy green plant is selected from the group consisting of lettuce, cauliflower, broccoli, cabbage, watercress, arugula, garlic, onion, leek, amaranth, swill chard, been, spinach, melon, cucumber, squash, basil, celery, cilantro, radish, radicchio, chicory, dill, rosemary, French tarragon, basil, Pennisetum, carrot, fennel, beans, peas, chickpeas, and lentils.

32. The method of any one of claims 23-31, wherein the mineral nutrient is selected from nitrogen, potassium, iron, magnesium, copper, calcium and sulfur.

33. An isolated Methylobacterium selected from the group consisting of LGP2022 (NRRL-B-68033), LGP2023 (NRRL-B-68034), and LGP2021 (NRRL-B-68032).

34. A composition comprising: (i) the isolated Methylobacterium of claim 33; and (ii) an agriculturally acceptable adjuvant, agriculturally acceptable excipient, or a combination thereof.


SEQUENCE LISTING (76 sequences)

SEQ ID NO: 1 - DNA, 300 bp, Methylobacterium
acggtcaccccacggactgggcgagtacctcaccggtgttctatcataacgccgagttagttttcgaccgtcccttatgcgatgtaccaccggtgtcggcagccgatttcgtcccaccgggagctggcgttccggttcagaccaccatcatcggtcacgatgtctggattggacacggggccttcatctcccccggcgtgactataggaaacggcgcgatcgtcggggcccaggcggtcgtcacaagagatgtcccaccctatgcggtagttgctggcgtccccgcgaccgtacgacgat

SEQ ID NO: 2 - DNA, 300 bp, Methylobacterium
ccaataaaagcgttggccgcctgggcaacccgatccgagcctaagactcaaagcgcaagcgaacacttggtagagacagcccgccgactacggcgttccagcactctccggctttgatcggataggcattggtcaaggtgccggtggtgatgacctcgcccgccgcaagcggcgaattactcggatcagcggccagcacctcgaccaagtgtcggagcgcgaccaaagggccacgttcgaggacgtttgaggcgcgaccagtctcgatagtctcatcgtcgcggcgaagctgcacctcga

SEQ ID NO: 3 - DNA, 300 bp, Methylobacterium
cgatggcaccgacctgccatgcctctgccgtccgcgccagaatggtaaagaggacgaagggggtaaggatcgtcgctgcagtgttgagcagcgaccagagaagggggccgaacatcggcatcaaacctcgattgccactcggacgcgaagcgcgtcttgaaggagggatggaagcgaaacggccgcagagtaaccgccgacgaaagattgcacccctcatcgagcaggatcggaggtgaaggcaagcgtgggttattggtaagtgcaaaaaatataatggtagcgtcagatctagcgttc

SEQ ID NO: 4 - DNA, 23 bp, Artificial Sequence
  Synthetic
cctcaccggtgttctatcataac

SEQ ID NO: 5 - DNA, 20 bp, Artificial Sequence
  Synthetic
ccgatgatggtggtctgaac

SEQ ID NO: 6 - DNA, 21 bp, Artificial Sequence
  Synthetic
cgtcccttatgcgatgtacca

SEQ ID NO: 7 - DNA, 22 bp, Artificial Sequence
  Synthetic
gatccgagcctaagactcaaag

SEQ ID NO: 8 - DNA, 21 bp, Artificial Sequence
  Synthetic
gaccaatgcctatccgatcaa

SEQ ID NO: 9 - DNA, 20 bp, Artificial Sequence
  Synthetic
aacacttggtagagacagcc

SEQ ID NO: 10 - DNA, 21 bp, Artificial Sequence
  Synthetic
aaggagggatggaagcgaaac

SEQ ID NO: 11 - DNA, 19 bp, Artificial Sequence
  Synthetic
ataacccacgcttgccttc

SEQ ID NO: 12 - DNA, 21 bp, Artificial Sequence
  Synthetic
cgcagagtaaccgccgacgaa

SEQ ID NO: 13 - DNA, 300 bp, Methylobacterium
gcaaaacgacctaatagttctacagcggcatgcgccaagtcagcgcggtgaacagtatacctgggagcaacttgtcctccgaaacccacataaaacaaattactcctggcagtgcccagtccatcaaaatcgaatacaatatttctcgaggaggcatctgtaatagcctgccaaagcaacaaagctatggcgccgttatgactttcattgcttctggtagacataaaataatatgccgatttgtgatcccaaatgtagaatattgccgcatcaattgcgccaagtttatttcggatcgat

SEQ ID NO: 14 - DNA, 300 bp, Methylobacterium
ggcgccaacggtatgatcgcatgattttcctgcggcatagcttgcgggaatggcgtatttggcgctctcctcaggaatttctaagggcatacgcaggaactctacagcacttttactggtattttgtagtgacagcggaggaggctggtgctcaaggtaatcgtgatgaagtgatccgggccattcggggcgcgtttctagtctttccaatccgcgccctgtaccacgtattacgccggaccggtctgcgccgcgccgccctcttgaccgccctaaatgtctaagagcgtctaacaaagc

SEQ ID NO: 15 - DNA, 300 bp, Methylobacterium
gacgatatcgctcatcttcactgcattgaagctggtgccgtactgcatagggatgaaaaagtgatgcggatagacggctgacgggaaagcgcctggtcgatcgaagactttgctgacgaggttgtggtagccccggatataggcatcgaaggccgggacgttgatcccatcctttgccttatcttgactggcgtcgtcgcgtgccgtcagaacgggcacgtcgcaggtcatcgaggccagcaccttgcggaacacctgcgttccgccgttgggattatcgacggcgaacgcggtggccgc

SEQ ID NO: 16 - DNA, 21 bp, Artificial Sequence
  Synthetic
gtcctccgaaacccacataaa

SEQ ID NO: 17 - DNA, 23 bp, Artificial Sequence
  Synthetic
ctaccagaagcaatgaaagtcat

SEQ ID NO: 18 - DNA, 22 bp, Artificial Sequence
  Synthetic
tctgtaatagcctgccaaagca

SEQ ID NO: 19 - DNA, 19 bp, Artificial Sequence
  Synthetic
ggctggtgctcaaggtaat

SEQ ID NO: 20 - DNA, 20 bp, Artificial Sequence
  Synthetic
acatttagggcggtcaagag

SEQ ID NO: 21 - DNA, 19 bp, Artificial Sequence
  Synthetic
atgaagtgatccgggccat

SEQ ID NO: 22 - DNA, 21 bp, Artificial Sequence
  Synthetic
ccgtactgcatagggatgaaa

SEQ ID NO: 23 - DNA, 21 bp, Artificial Sequence
  Synthetic
taaggcaaaggatgggatcaa

SEQ ID NO: 24 - DNA, 21 bp, Artificial Sequence
  Synthetic
ttgctgacgaggttgtggtag

SEQ ID NO: 25 - DNA, 300 bp, Methylobacterium
caactatgtagacccgacggtgcgatttcacttcgcaaagccgcagggcagcacccttgcgctcaatgttgacgccagcgtgatctatactattaccgtcacgcacacgcagggcggcgtacagattcatcgcgagagtaagaaccaccatcagaccatcacgcgcagcgacctgagcaagcagttcggcgttggtgtggccgaccagctgacgcgcgatcaggtcatgaaggtgatcgagtcggcatttcgcgacgctacccgctaagatcggcgcccacgaaacgctacgagactagg

SEQ ID NO: 26 - DNA, 300 bp, Methylobacterium
agccggcatcttgttcaaggcgctcacctcgacgccgacgctgtaggcgacttgagagggcgtctcatatgaacgaagcatcttcgcgtagagaaccttcttgttctcctgcgtgatgttcgctttgcagacgttgactgccgccatgaacgccgaagccttgcgcgcttcatcgtaatcgcctgcgaaggcgggtagtgaaaagcttagtgcaatggcaaacacagccgccgaacgtcgcatggtatccgtccccgattgacggcagtgccgccatatctcggctttagcagagctgat

SEQ ID NO: 27 - DNA, 300 bp, Methylobacterium
aacctgcgccggccgaggtttcgcgagccgtcgccacgggcaacgcctcgcccgcgatgtgcaaaaaagtccccggcacttcgcgccgtcgtccgatccacgaccgcgaatttctcaacgagtacaaggtgcttatgggagatccgagcgtccgtcccggagcccgagaccgcgcggcccgagtaataggcgaaaaagactcctactcctcgggcttctcgggccccctcagcaacatctacgcttgccgcccatcaccctggcgggagatcagcgacgagacacaggcccacttcgccc

SEQ ID NO: 28 - DNA, 22 bp, Artificial Sequence
  Synthetic
ttgacgccagcgtgatctatac

SEQ ID NO: 29 - DNA, 22 bp, Artificial Sequence
  Synthetic
gtgatggtctgatggtggttct

SEQ ID NO: 30 - DNA, 19 bp, Artificial Sequence
  Synthetic
tattaccgtcacgcacacg

SEQ ID NO: 31 - DNA, 24 bp, Artificial Sequence
  Synthetic
cttcgcgtagagaaccttcttgtt

SEQ ID NO: 32 - DNA, 22 bp, Artificial Sequence
  Synthetic
cttcgcaggcgattacgatgaa

SEQ ID NO: 33 - DNA, 20 bp, Artificial Sequence
  Synthetic
cgtgatgttcgctttgcaga

SEQ ID NO: 34 - DNA, 23 bp, Artificial Sequence
  Synthetic
ccgcgaatttctcaacgagtaca

SEQ ID NO: 35 - DNA, 21 bp, Artificial Sequence
  Synthetic
gcccgaggagtaggagtcttt

SEQ ID NO: 36 - DNA, 20 bp, Artificial Sequence
  Synthetic
aggtgcttatgggagatccg

SEQ ID NO: 37 - DNA, 300 bp, Methylobacterium
agtcattgatcaagcaacccctattgagttggatatcgaaggatcaaggtcgcgtcaatagatgcatctatcaggccaaatgtcgcttttcaagaatggctctttcgaagctatctttataatcgctcgccattctctcattaccaaaatcgaccttaactagctcgacattgatgcgagcagctccggcaaacgaggagagattgaccttaaaggaattgaacgcctcaagcaattcagacacattaccaggagtgctatagcaacaaccagacccatatcggtcaataacctctttta

SEQ ID NO: 38 - DNA, 300 bp, Methylobacterium
cgcaaaacgatttatcactgccatcttgttgtttgataacccttttttaccagacgttatgctgggcgagaaagaggactagcagatcggagcggtatcgcgatttttcggtagttcgcgcctacaacaggataagatccgatagtgaagcaacatggctgttttttgatttgtaagtcagcaacttaagcagccagcctatctgccgtcgcagacgcttgaggcatcgggcagcatcttagaaaaggtggcagtaattgccacagcggaacgtagcggcacggataagcacgcagggtc

SEQ ID NO: 39 - DNA, 300 bp, Methylobacterium
cccatctggacccaatatccccttcatcgacaattcccgagtaagtgtgggttcgaggatttcgcgaaacagccttgttcgttcctccggccttaaaattggcgtgccgtcgggagatcgataggcatcccttacctgcctttcgaccgccggcacacgcgcgccggtcgtcgtgttcacggccacggaatggacgaaggtgcgccgctcatttcgctcgtttgccgtctccaccatccaggaggccagcaggacggtttcgtctcgaccgccggtcacacacaccgcaagggactcagg

SEQ ID NO: 40 - DNA, 22 bp, Artificial Sequence
  Synthetic
tcgctcgccattctctcattac

SEQ ID NO: 41 - DNA, 22 bp, Artificial Sequence
  Synthetic
aggtcaatctctcctcgtttgc

SEQ ID NO: 42 - DNA, 18 bp, Artificial Sequence
  Synthetic
tcgacattgatgcgagca

SEQ ID NO: 43 - DNA, 22 bp, Artificial Sequence
  Synthetic
ttcgcgcctacaacaggataag

SEQ ID NO: 44 - DNA, 23 bp, Artificial Sequence
  Synthetic
cagataggctggctgcttaagtt

SEQ ID NO: 45 - DNA, 18 bp, Artificial Sequence
  Synthetic
tccgatagtgaagcaaca

SEQ ID NO: 46 - DNA, 24 bp, Artificial Sequence
  Synthetic
gagtaagtgtgggttcgaggattt

SEQ ID NO: 47 - DNA, 23 bp, Artificial Sequence
  Synthetic
aggtaagggatgcctatcgatct

SEQ ID NO: 48 - DNA, 18 bp, Artificial Sequence
  Synthetic
cggaggaacgaacaaggc

SEQ ID NO: 49 - DNA, 300 bp, Methylobacterium
acctgctaaaatcacgtcctctcagattgaaaaatcattgaagaaacgtgtcgaacgattgccggggattatgacgttagatcaattgaaaaatacaagctttgaaattgagttacagccaaaagatgccccggatccggacccatcagacttcggtggctagttcgagccaaactcgaacgtcgccatggcgcgcaagtcgcaataccatttcacagcgcagcggttatttcgttgtacactgtagcaatgcgtcggcttgcgcgcttccgctggcgatcaaaggtccgccgatttacg

SEQ ID NO: 50 - DNA, 300 bp, Methylobacterium
tcccgaacatacaatggaggaagcgtgtggtaggccaatttgtaacgaaatatggcatcggtcacggctctctcaataaattcgatctcaagtcttctgaacgagcatgcctcatccttatcctgagcgaacgcctgccagtttgcagtcattccaacatacatagccaaaaaggcgaggtagaccttcatacgggcacctcaatcgtccccattcgttcaagctccttcaagataacagccgcaccacattgctgagatcgaagattcggatcaaatattccatcaaatttatactttc

SEQ ID NO: 51 - DNA, 300 bp, Methylobacterium
gcatcctttgcgctcgcaggcctaaggtcaagcccggttacttcgtttggtagaacgaggtagacgatgcctagtcttaaggtggcccatgttaaccaacagggccagaacatgattatagttccgttagatgccaacttcggttacaaaaccgatggtgagcagtccgacatcatgttcgaaatacaggacgcggcgcggtccgccggtcttgcgggtgccgtagtagcgttctggcagtcaggtggacaaacccgtttccggggcccggctccgtggcacccattccttcgcagcctc

SEQ ID NO: 52 - DNA, 22 bp, Artificial Sequence
  Synthetic
gcgcaagtcgcaataccatttc

SEQ ID NO: 53 - DNA, 21 bp, Artificial Sequence
  Synthetic
cgtaaatcggcggacctttga

SEQ ID NO: 54 - DNA, 19 bp, Artificial Sequence
  Synthetic
cgcagcggttatttcgttg

SEQ ID NO: 55 - DNA, 22 bp, Artificial Sequence
  Synthetic
acgagcatgcctcatccttatc

SEQ ID NO: 56 - DNA, 21 bp, Artificial Sequence
  Synthetic
cgattgaggtgcccgtatgaa

SEQ ID NO: 57 - DNA, 20 bp, Artificial Sequence
  Synthetic
tgccagtttgcagtcattcc

SEQ ID NO: 58 - DNA, 23 bp, Artificial Sequence
  Synthetic
cccggttacttcgtttggtagaa

SEQ ID NO: 59 - DNA, 24 bp, Artificial Sequence
  Synthetic
cgaagttggcatctaacggaacta

SEQ ID NO: 60 - DNA, 20 bp, Artificial Sequence
  Synthetic
tggcccatgttaaccaacag

SEQ ID NO: 61 - DNA, 300 bp, Methylobacterium
gcccttctgtcaggcgatattgtataatggcgttgccccaatagaagcagccattcgtgcgagggcagcagcgacgctaggtcgaaagagcatcctaatctcgatcaagatgcgactgagatttctgatgaaaatatctagacacaagcaaagctggtgaaattacaacgatcatggcgacaattgcggccaattcggccggaacttgaaggaacataaaaatgaatattacaaatataccgcaaagcatgtagagttgctacaccaagggtcgggacgtccaaaaaaactcactgagga

SEQ ID NO: 62 - DNA, 300 bp, Methylobacterium
ggaacataaaaatgaatattacaaatataccgcaaagcatgtagagttgctacaccaagggtcgggacgtccaaaaaaactcactgaggaagtcgactggaagcacgaggcgccccccccaggagcggggcgaccggcaagggggcccgcaattgtcgccatgatcgaccagcttaggtaggatcctctttcgacctaacgaatggctgcttctattggggcaacgccattatacaatatcgcctgaccatctggaacgcggcccggtccaccggcaggttggcgacgacagcgtcggag

SEQ ID NO: 63 - DNA, 300 bp, Methylobacterium
cggcgtcgaccagccgggcgaactgcttgggcatgctctcccgcgacgccggccacagccgcgtccccgtccctccgcacaggatcatcgggtggatttgaaaggcaaaacgggacatcaggataggccgctcaggcgttggcgctgaggcgcttgatgtcggcgtcgaccatctcggtgatcagcgcctcgaggctggtctcggcctcccagccgaaggtcgccttggccttggcggggttgcccagcagcacctcgacctctgccggccggaacagcgccgggtcgacgatcaggtgg

SEQ ID NO: 64 - DNA, 300 bp, Methylobacterium
ctggacatgcgcccaccccggccaagtccgaccgcaccggcaaccgctcctgtagtcgtcgtcatcgttctcacccctgaggcggagaccgtccgctaacggggtgtctcaagcaaccgtggggcggaggaacacgcacgtagtcgcgtttcaaggttcgcacgaacgcctcggccatgccgttgctctgcgggctctccagcggcgtcgtttttggcaccaaaccaaggtcgcgggcgaagcggcgcgtgtcgcggggactgtcaggaatttcgtgtgggggcggccatagtggatccg

SEQ ID NO: 65 - DNA, 300 bp, Methylobacterium
ggaaatcggcttcaagtacgacgtcacgccggccatgcaggtcacgggtgcactgttcaatctcgagcgcgacaaccagccgttcccctcgaacgtggagtccggcctcgtccttggcgcaggtcagacacgcacccagggcgcggaaatcggcctggccggctatctaaccgattggtggcaggtctttggcggctacgcttataccgaggcacgcgtactctcgccactggaagacgatggagacgtgatcgcagcaggtaatctcgtcggcaacgttccgctaaatactttcagtct

SEQ ID NO: 66 - DNA, 300 bp, Methylobacterium
cggcctggccggctatctaaccgattggtggcaggtctttggcggctacgcttataccgaggcacgcgtactctcgccactggaagacgatggagacgtgatcgcagcaggtaatctcgtcggcaacgttccgctaaatactttcagtctgttcaacaagttcgatatcaacgagaatttctccgttgctctgggctattactatcaggatgccagctttgcctcctcagacaatgcagtgcgtttgccaagttattcgcggttcgatggcgggttgttctatcgattcgacgagttgac

SEQ ID NO: 67 - DNA, 300 bp, Methylobacterium
acgttccgctaaatactttcagtctgttcaacaagttcgatatcaacgagaatttctccgttgctctgggctattactatcaggatgccagctttgcctcctcagacaatgcagtgcgtttgccaagttattcgcggttcgatggcgggttgttctatcgattcgacgagttgacacgcgttcagcttagcgtcgagaacattttcgacaggcgttacatcatcaactccaacaacaacaacaacctcacgcctggcgcgccgagaacagtccgcgtgcaattgatcgctcggttctaaa

SEQ ID NO: 68 - DNA, 300 bp, Methylobacterium
agcccacaagcctgatgcacttaactacatcctctaatgtcgcgccaatttgcttggcggcaggggatgttgtatcgtcataggcttgtctaaccggaacttgtttgccaatctctttggcgatcgcaaccgccatctcgtgttcgtcaaccatgtgcgcgttcctctaattgcactcatggtgccacgtgcacctccgatcgtctcgtgtctagaatgaaggtgggaacaaccttacacaggctttcgcgacgcgcgaatttctggtttctccgcctcggatgtgggtttgagcgcttc

SEQ ID NO: 69 - DNA, 300 bp, Methylobacterium
cttttcatttgtcatgatctcgaccaaggtattcacggcaagctcggtctgttgcttagcaagtgcctgaacttcgcgaacgatcggctctcgacccttcgggttcgagacctgtcccttttgaaaaccacgtgccctacacttttcgggatcaaggtgcgggttggctttggtcaaaattctctggcgtcccattacacgccctccgcatcatcgttcccgcgaacgatctgacccccgacttccgcgaggaagcgtgtggcgtgatcctcgaagcggaatgccacctcgaactgttcc

SEQ ID NO: 70 - DNA, 300 bp, Methylobacterium
cagcagcaagcagatcgttgaaaaccgcttgaaccgcatcttgatcgggaccggaaccaatcaggtcatctaggtaaaccgagacgtaaactcgtttgcgctcggcatctttcagaacgtccgtgatgccagaccgcattagtaccatcgtcgccaaggcgggcgactgaacgaagccgatcggcagagagtaacggggaccgcccctaatcgggttgcgaacgcaagaccacttagcaaaggttcgagcacggccgaacttcgcatggtggagagccgcggcaacacggttccgtgata

SEQ ID NO: 71 - DNA, 300 bp, Methylobacterium
tagacattccaacaaaccggcaagaggctcgtcctcactcgaggatttgttgggacttgcatgatgtcgaagcggagccgttatgacctgggtgcgatcatgcgccgagcatgggagatggctcgggaggcggcattcgcggttggcgagcgggcacggactcaccttgctgccgcgatgcgcagcgcgtgggccgaagccaagttggcactcgcgcccacgaagacggagcaggatcgtctctctccgagcgacatgatcggacatgaggacgcctaccaaggccgggttctaaaatat

SEQ ID NO: 72 - DNA, 300 bp, Methylobacterium
aagatggatacgacaagcgcgattacattatttgcgaaatagatggacaaataaaagacaaaggactgatgtatttccttaaatctggacaagttgacctctttcacatagaagtcaccactccctttgggacaatttggtgtcacgaaaacatagaggccgaacttcttagctgaattatcgcgctccgggttcttatgcggctgagtgaagcgcgggacagcttgcgagcagggccgccaatggcagccgggatgacacaatgctcggtctcccgacgcttcttcaatcgggagcgct

SEQ ID NO: 73 - DNA, 299 bp, Methylobacterium
agctgaattatcgcgctccgggttcttatgcggctgagtgaagcgcgggacagcttgcgagcagggccgccaatggcagccgggatgacacaatgctcggtctcccgacgcttcttcaatcgggagcgcttcgcagcccggggcggcgcgctcatgcgtcacgacctgggccctgcgcaccttcgcggccccgccgtcccggcagatccctgatgccccaagtgggcggccactccatcaaagaaccccggcctgtggcagatctcgtaggcataccgaggttccgcagtgcccccacc

SEQ ID NO: 74 - DNA, 300 bp, Methylobacterium
accgaaggcgtccccggacacgaaggcctgaaacaccatatctgtggcgatcaggccgacgtggtcgcggacttcaactggcagagaatgccaggccgcttcgatttcagatgatactggtacggacataggagcggcttagctttctcagtgcaaatgtgattgattccggctcaaaaatgatcttgatcggacgagacgttttcaatccatgtcgtgttgccatcgccgatcggtgcgtcaagagacagatggcgccgaccgtagatacgcgttcgggttgcccgcaccgcttctcca

SEQ ID NO: 75 - DNA, 300 bp, Methylobacterium
ggaggtgtgatctgatgatgtgctggatgaaattggcggtcgagcacttgttcagcttggccagctcgacgagatcggcgtgatgctcggcgtcgatcaggatgttcagcgagaccggacgtacgcaggacttggtattagcgccgttgcgcatcagcttgcagccttgctctgcttctcagcgtgccgcgtcaggatgaccctgatgtagctgttgaggttgatgccgtaatagcctgcggactctgtgagatcccggcgaagatcgtcggcgagggtcaggcggatggtgctggtcgg

SEQ ID NO: 76 - DNA, 300 bp, Methylobacterium
aagtaaccgctcaacatgatcttcagcatgttgtccaacagcaggagaatacatgtaattcaccatgaccggcaagctgcgactggccattgcttccaccgcttgaatgtagcgatcgaatttcgcaaaatcagggtggaatgaaaatatcgaaccaaactgcgagccttgaatccgttctgcaaaattatcgaaaaattttcttggccgactgccgttcgaaaacattcttacgtttacatgcggcccgcctgaaacaagacagtctaccagctctgggaaatgggggtgaagggtcgg